(19) United States
(12) Patent Application Publication
(10) Pub. No.: US 2026/0126391 A1
(43) Pub. Date: May 7, 2026

(54) GAS ANALYSIS BASED ON RAMAN SPECTROSCOPY

(71) Applicant: Provincial Health Services Authority, Vancouver (CA)

(72) Inventors: Maryam SHIRMOHAMMAD, Irvine, CA (US); Michael Anthony SHORT, Coquitlam (CA); Haishan ZENG, Vancouver (CA)

(21) Appl. No.: 19/429,561

(22) Filed: Dec. 22, 2025

Related U.S. Application Data
(63) Continuation of application No. PCT/CA2024/050885, filed on Jun. 28, 2024.
(60) Provisional application No. 63/523,728, filed on Jun. 28, 2023.

(51) Int. Cl. G01N 21/65; G01N 1/38; G01N 33/00

(52) U.S. Cl. CPC G01N 21/65; G01N 1/38; G01N 33/0016

(57) ABSTRACT

A method for detecting a presence of at least one constituent of an analyte gas is based on Raman scattering. The method comprises: mixing a buffer gas with the analyte gas; exciting the mixture with a laser excitation beam; detecting scattered photons resulting from interaction between photons of the laser excitation beam and molecules of the analyte gas to thereby determine a spectral content of the scattered photons; and analyzing the spectral content to determine the presence of the at least one constituent.

### CROSS-REFERENCE TO RELATED APPLICATIONS

This application is a continuation of Patent Cooperation Treaty (PCT) application No. PCT/CA2024/050885 having an international filing date of 28 Jun. 2024, which in turn claims priority from, and for the purposes of the United States of America the benefit under 35 U.S.C. § 119 in relation to U.S. application No. 63/523,728 filed 28 Jun. 2023. All of the applications in this paragraph are hereby incorporated herein by reference.

### FIELD

This invention relates to systems and methods for gas analysis based on Raman scattering. Aspects of the invention provide systems and methods for detecting a presence of at least one constituent of an analyte gas based on Raman scattering. Aspects of the invention relate to enhancing Raman scattering for gas analysis.

### BACKGROUND

Raman spectroscopy is a versatile chemical analytical technique having a wide range of applications. Raman spectroscopy is based on the phenomenon of Raman scattering, which refers to the interaction (i.e. inelastic scattering of photons) of light with a material (e.g. vibrational or rotational modes of a molecule of the material).

Molecules vibrate with different modes known as normal modes or natural frequencies. Each of these vibrational modes has a unique fundamental frequency. Rotational modes of a molecule refer to the quantized rotational motion of the molecule around its center of mass and are characterized by discrete energy levels determined by the molecule's moment of inertia and rotational constants. These vibrational and rotational modes are specific to the chemical bonds present in the particular molecules. As a result, Raman scattering may yield detailed information about the chemical structure of the molecules, thereby enabling a “fingerprint” type of analysis to identify molecules in a sample.

One application of Raman spectroscopy is gas analysis, where Raman spectroscopy is applied to determine compositions (i.e. one or more constituents) of a gas mixture. Raman gas analyzers based on spontaneous Raman scattering have been used in petrochemical industries, environmental studies, analysis of exhaled breath, etc.

There is a general desire to analyze exhaled breath. Raman spectroscopy for analyzing exhaled breath has the advantages of lower cost, more compact size and faster turnaround time compared to other common techniques of breath analysis, such as mass spectrometers. However, Raman gas analysis of exhaled breath is also challenging due to the low-molecular-number densities of the analyte gas and the intrinsically inefficient spontaneous Raman scattering due to low inelastic Raman scattering cross-sections. For example, approximately only one in a million interactions between photons of an excitation light and molecules of an analyte gas results in spontaneous Raman scattering. Consequently, there is a desire to provide Raman enhancement techniques to improve the practical utility of Raman scattering based gas analysis and, for example, Raman scattering based analysis of exhaled breath.

Various Raman enhancement techniques have been developed and proposed to improve Raman signal intensity.

Some Raman enhancement techniques strive to increase the Raman interaction pathlength, which refers to the distance over which the incident excitation light interacts with the sample. An increase in interaction pathlength allows more photons of the excitation light to interact with molecules of the sample, leading to a higher probability of inelastic scattering event to thereby enhance Raman scattering.

One enhancement technique that strives to increase the interaction pathlength is cavity-enhanced Raman spectroscopy. In cavity-enhanced Raman spectroscopy, the effective pathlength for Raman interaction is increased through multiple-beam passing by using specialized reflection mirrors in a gas cell. However, the specialized reflection mirrors are costly to manufacture due to the requirement of precision manufacturing. Moreover, achieving and maintaining proper alignment of the lasers and the optical components (e.g. the specialized reflection mirrors) is tedious and complicated.

Another example enhancement technique that strives to increase the interaction pathlength is fiber-enhanced Raman spectroscopy (FERS). FERS utilizes specialized hollow-core optical fibers to significantly lower attenuation loss thereby enabling longer fiber length for light gas interaction. Example hollow-core optical fibers include bandgap transmission hollow-core fibers and anti-resonant hollow-core fibers. One type of bandgap transmission hollow-core fibers is the hollow-core photonic-crystal fiber (HCPCF). The use of these hollow-core fibers increases the light-gas interaction pathlength without significant attenuation of the Raman signal.

Nonlinear enhancement techniques such as coherent anti-Stokes Raman scattering (CARS) and double-beam stimulated Raman scattering (SRS) have also been proposed. These enhancement techniques utilize multiple pump and Stokes lasers to coherently drive the vibrational or rotational modes of gas molecules to achieve Raman intensities of orders of magnitude greater than spontaneous Raman scattering. However, in both CARS and dual-beam SRS systems, proper alignment of two or more lasers is essential in obtaining good results. Achieving and maintaining proper alignment of the lasers are complicated in practice. Moreover, a tunable Stokes laser is needed for both CARS and dual-beam SRS, making these systems relatively more costly.

Different Raman enhancement techniques can also be combined. For example, a FERS-based SRS system has been proposed using a continuous wave (CW) laser as the Raman excitation pump source. Major components of exhaled-breath gas were successfully identified by such a system. However, the system was insufficiently sensitive to identify trace amounts of minor components in exhaled breath, such as volatile organic compounds (VOCs). Some VOCs can be used as biomarker for cancer detection and detection of such VOCs can aid cancer screening.

Therefore, there is an ongoing desire for cost-effective and simple-to-implement systems and methods for gas analysis based on Raman spectroscopy that is capable of detecting even trace amounts of minor components in an analyte gas. There is also an ongoing desire for cost-effective and simple-to-implement Raman enhancement techniques that may generate significant (e.g. up to orders of magnitude higher) enhancement of Raman intensities.

### SUMMARY

One aspect of the invention provides a method for detecting a presence of at least one constituent of an analyte gas based on Raman scattering. The method comprises: mixing a buffer gas with the analyte gas; exciting the mixture with a laser excitation beam; detecting scattered photons resulting from interaction between photons of the laser excitation beam and molecules of the analyte gas to thereby determine a spectral content of the scattered photons; and analyzing the spectral content to determine the presence of the at least one constituent.

An interaction pathlength between the photons of the excitation beam and the molecules of the analyte gas in the mixture may be greater than an interaction pathlength between the photons of the excitation beam and the molecules of the analyte gas in an absence of the buffer gas.

Determining the presence of the at least one constituent may be based on identifying at least one peak in the spectral content corresponding to the at least one constituent.

The magnitude of the at least one peak may be greater than a magnitude of the at least one peak in the absence of the buffer gas.

The method may comprise increasing the temperature of the mixture relative to a temperature of the ambient environment. Increasing the temperature of the mixture relative to the temperature of the ambient environment may increase the average speed of molecules of the mixture compared to an average speed of the molecules of the mixture at the temperature of the ambient environment.

The buffer gas may have a buffer gas partial pressure and the analyte gas may have an analyte gas partial pressure wherein the buffer gas partial pressure is different from (preferably greater than) the analyte gas partial pressure.

A ratio of the buffer gas partial pressure to the analyte gas partial pressure may be in a range of about 0.01 to about 1000.

The method may comprise holding the analyte gas partial pressure constant and increasing the buffer gas partial pressure relative to the analyte gas partial pressure.

The method may comprise mixing the buffer gas and the analyte gas in a gas cell.

Exciting the mixture with the laser excitation beam may comprise directing the laser excitation beam into the gas cell. The gas cell may comprise a hollow-core fiber. The hollow-core fiber may comprises a hollow core which permits permeation of fluid in the hollow core. Mixing the buffer gas with the analyte gas in the gas cell may comprise introducing the buffer gas and the analyte gas into the hollow core of the hollow-core fiber.

A diameter of the hollow core may be in a range of about 2 μm to about 50 μm. The hollow-core fiber may have a longitudinal length in a range of about 0.5 m to about 20 m.

The hollow-core fiber may comprise a band-gap transmission hollow-core fiber.

The hollow-core fiber may comprise a hollow-core photonic crystal fiber.

The hollow-core fiber may comprise an anti-resonant hollow-core fiber.

The method may comprise directing the laser excitation beam into the hollow core of the hollow-core fiber.

The laser excitation beam may be confined by the hollow-core fiber to the hollow core.

The method may comprise mixing the buffer gas and the analyte gas at an analyte source.

Mixing the buffer gas and the analyte gas at the analyte source may comprise supplying a preparation gas to the analyte source wherein the preparation gas comprises the buffer gas at a buffer gas mole fraction. The analyte source may comprise breath (e.g. exhaled) of a human or other animal and the preparation gas may be safe for inhalation by the human or other animal.

The buffer gas mole fraction in the preparation gas may be in a range of about 75% to about 85%.

The laser excitation beam may comprise a pulsed single laser excitation beam. The pulsed single laser excitation beam may have a pulse duration in a range of about 0.01 ns to about 15 ns. The pulsed single laser excitation beam may have a pulse repetition rate in a range of about 0.1 Hz to about 100 MHz. The pulsed single laser excitation beam may have a spectral bandwidth less than 20 cm−1.

The buffer gas may comprise an inert gas.

The buffer gas may comprise a noble gas.

The buffer gas may comprise Helium.

The buffer gas may comprise Nitrogen gas.

The spectral content may contain information about Raman intensity over a range of wavenumbers.

Another aspect of the invention provides a method for detecting a presence of at least one constituent of an analyte gas based on Raman scattering. The method comprises: supplying the analyte gas to a hollow core of at least one hollow-core micro-structured fiber; exciting the analyte gas with a pulsed single optical excitation beam above a threshold pump power, the pulsed single optical excitation beam directed into the hollow core and confined by micro-structures of the at least one fiber to the hollow core to thereby interact with the analyte gas; detecting scattered photons resulting from interaction between the photons of the excitation beam and the molecules of the analyte gas to thereby determine a spectral content of the scattered photons; and analyzing the spectral content to determine the presence of the at least one constituent.

The method may comprise supplying a buffer gas to the hollow core. Exciting the analyte gas with the excitation beam may comprise exciting a mixture of the buffer gas and the analyte gas in the hollow core with the excitation beam.

An interaction pathlength between the photons of the excitation beam and the molecules of the analyte gas in the mixture may be greater than an interaction pathlength between the photons of the excitation beam and the molecules of the analyte gas in an absence of the buffer gas.

Determining the presence of the at least one constituent may be based on identifying at least one peak in the spectral content corresponding to the at least one constituent.

The magnitude of the at least one peak may be greater than a magnitude of the at least one peak in the absence of the buffer gas.

The method may comprise increasing the temperature of the mixture relative to a temperature of the ambient environment. Increasing the temperature of the mixture relative to the temperature of the ambient environment may increase the average speed of molecules of the mixture compared to an average speed of the molecules of the mixture at the temperature of the ambient environment.

The buffer gas may have a buffer gas partial pressure in the mixture. The analyte gas may have an analyte gas partial pressure in the mixture. The buffer gas partial pressure may be different from (preferably greater than) the analyte gas partial pressure.

A ratio of the buffer gas partial pressure to the analyte gas partial pressure may be in a range of about 0.01 to about 1000.

The method may comprise holding the analyte gas partial pressure constant and increasing the buffer gas partial pressure relative to the analyte gas partial pressure.

The method may comprise mixing the buffer gas and the analyte gas in a gas cell. Supplying the analyte gas and the buffer gas to the hollow core may comprise supplying the mixture to the hollow core.

The hollow core may permit permeation of fluids contained therein.

A diameter of the hollow core may be in a range of about 2 μm to about 50 μm. The hollow-core micro-structured fiber may have a longitudinal length in a range of about 0.5 m to about 20 m.

The hollow-core micro-structured fiber may comprise a band-gap transmission hollow-core fiber.

The hollow-core micro-structured fiber may comprise a hollow-core photonic crystal fiber.

The hollow-core micro-structured fiber may comprise an anti-resonant hollow-core fiber.

The laser excitation beam may be confined by the hollow-core micro-structured fiber to the hollow core.

The method may comprise mixing the buffer gas and the analyte gas at an analyte source. Supplying the analyte gas and the buffer gas to the hollow core may comprise supplying the mixture from the analyte source to the hollow core.

Mixing the buffer gas and the analyte gas at the analyte source may comprise supplying a preparation gas to the analyte source. The preparation gas may comprise the buffer gas at a buffer gas mole fraction. The analyte source may comprise breath (e.g. exhaled) of a human or other animal. The preparation gas may be safe for inhalation by the human or other animal.

The buffer gas mole fraction in the preparation gas may be in a range of about 75% to about 85%.

The pulsed single optical excitation beam may comprise a pulsed single laser excitation beam.

The pulsed single laser excitation beam may have a pulse duration in a range of about 0.01 ns to about 15 ns. The pulsed single laser excitation beam may have a pulse repetition rate in a range of about 0.1 Hz to about 100 MHz. The pulsed single laser excitation beam may have a spectral bandwidth less than 20 cm−1 .

The buffer gas may comprise an inert gas.

The buffer gas may comprise a noble gas.

The buffer gas may comprise Helium.

The buffer gas may comprise Nitrogen gas.

The spectral content may contain information about Raman intensity over a range of wavenumbers.

Another aspect of the invention provides a system for detecting a presence of at least one constituent of an analyte gas based on Raman scattering. The system comprises: a gas cell for holding gas; one or more gas supplies in fluid connection with the gas cell for supplying the analyte gas and a buffer gas to the gas cell; an optical excitation source optically connected to the gas cell for emitting an optical excitation beam into the gas cell, the optical excitation beam propagating through the gas cell to interact with the analyte gas; a detector optically connected to the gas cell for detecting scattered photons resulting from interaction between the excitation beam and the analyte gas to thereby determine a spectral content of the scattered photons; and a processor in communication with the detector for analyzing the spectral content to determine the presence of the at least one constituent.

The system may comprise any of the features, combinations of features and/or sub-combinations of features of any of the other aspects recited herein.

Another aspect of the invention provides a system for detecting a presence of at least one constituent of an analyte gas based on Raman scattering. The system comprises: at least one hollow-core micro-structured fiber, the at least one fiber comprising a hollow core; an analyte gas supply in fluid connection with the at least one fiber for supplying the analyte gas to the hollow core; an optical excitation source optically connected to the at least one fiber for emitting a pulsed single optical excitation beam into the hollow core above a threshold pump power, the pulsed single optical excitation beam confined by micro-structures of the at least one fiber to the hollow core to thereby interact with the analyte gas therein; a detector optically connected to the at least one fiber for detecting scattered photons resulting from interaction between photons of the pulsed single excitation beam and molecules of the analyte gas; and a processor in communication with the detector for analyzing the spectral content to determine the presence of the at least one constituent.

The system may comprise any of the features, combinations of features and/or sub-combinations of features of any of the other aspects recited herein.

It is emphasized that the invention relates to all combinations of the above features, even if these are recited in different claims.

Further aspects and example embodiments are illustrated in the accompanying drawings and/or described in the following description.

### BRIEF DESCRIPTION OF THE DRAWINGS

1 The accompanying drawings illustrate non-limiting example embodiments of the invention.

FIG. 1 is a schematic diagram of a system for gas analysis based on collision-enhanced Raman scattering according to an example embodiment.

FIG. 2 is a flowchart of a method for detecting a presence of at least one constituent of an analyte gas based on collision-enhanced Raman scattering according to an example embodiment.

FIG. 3 is a schematic diagram of a system for gas analysis based on collision-enhanced Raman scattering according to another example embodiment.

FIG. 4 is a schematic diagram of a system for gas analysis based on single-beam stimulated Raman scattering and fiber-enhanced Raman scattering according to an example embodiment.

FIG. 5 is a schematic diagram of a system for gas analysis based on single-beam stimulated Raman scattering and fiber-enhanced Raman scattering according to another example embodiment.

FIG. 6 is a flow chart of a method for detecting a presence of at least one constituent of an analyte gas based on single-beam stimulated Raman scattering and fiber-enhanced Raman scattering according to an example embodiment.

FIGS. 7A and 7B are plots showing the spectral content of Raman scattering at different pressures according to experimental data.

FIG. 7C is a plot showing the relationship between Raman intensity and pressure of the analyte gas with Hydrogen gas being the analyte gas according to experimental data.

FIG. 8A is a plot showing the spectral content of Raman scattering at high buffer gas pressure with N₂ being the buffer gas and Hydrogen gas being the analyte gas according to experimental data.

FIG. 8B is a plot showing the relationship between Raman intensity and pressure ratio between buffer gas and the analyte gas with N₂ being the buffer gas and Hydrogen gas being the analyte gas according to experimental data.

FIG. 9A is a plot showing the spectral content of Raman scattering at high buffer gas pressure with Helium being the buffer gas and Hydrogen gas being the analyte gas according to experimental data.

FIG. 9B is a plot showing the relationship between Raman intensity and pressure ratio between buffer gas and the analyte gas with Helium being the buffer gas and Hydrogen gas being the analyte gas according to experimental data.

FIG. 10 is a plot comparing the efficiency of Raman intensity enhancement among three different set-ups according to experimental data.

FIGS. 11A-D are plots showing the spectral content of Raman scattering with propene being the analyte gas at different buffer gas partial pressures of the buffer gas with Helium being the buffer gas according to experimental data.

### DETAILED DESCRIPTION

Throughout the following description, specific details are set forth in order to provide a more thorough understanding of the invention. However, the invention may be practiced without these particulars. In other instances, well known elements have not been shown or described in detail to avoid unnecessarily obscuring the invention. Accordingly, the specification and drawings are to be regarded in an illustrative, rather than a restrictive sense.

Aspects of the invention relate to systems and methods for detecting a presence of at least one constituent of an analyte gas based on Raman scattering. In some particular embodiments, the analyte gas is exhaled breath.

A first aspect of the invention relates to systems and methods for gas analysis based on collision-enhanced Raman scattering. Collision-enhanced Raman scattering is facilitated by mixing one or more buffer gases with an analyte gas to form a gas mixture for interaction with an optical excitation light. A partial pressure of the buffer gas may be different from a partial pressure of the analyte gas. The partial pressure of the buffer gas may be higher than the partial pressure of the analyte gas in the gas mixture. The buffer gas may include, but is not limited to, an inert gas, a noble gas, Helium, Nitrogen gas, etc.

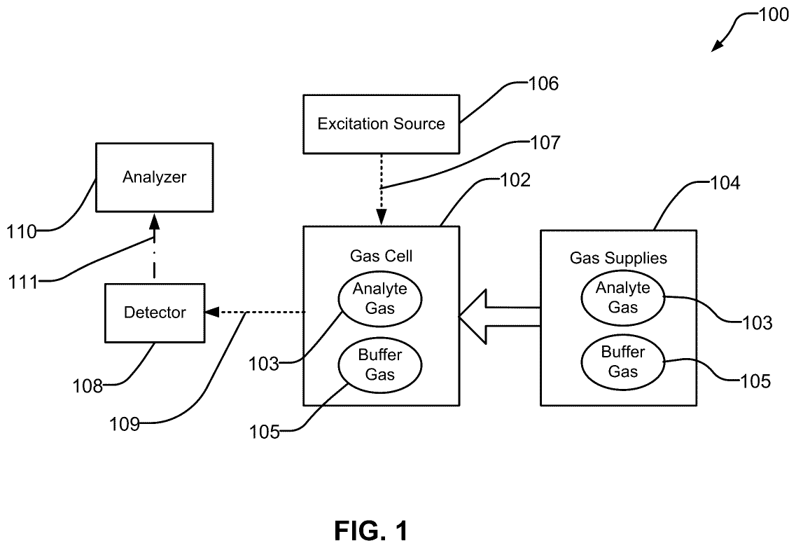

FIG. 1 is a schematic diagram of a system 100 for gas analysis based on Raman scattering according to an example embodiment. System 100 may be applied to determine a presence of at least one constituent of an analyte gas 103.

System 100 comprises one or more gas supplies 104 in fluid connection with a gas cell 102 for supplying an analyte gas 103 and a buffer gas 105 to a gas cell 102. Gas supplies 104 may comprise any suitable devices for supplying or providing analyte gas 103 and buffer gas 105 to gas cell 102. Gas supplies 104 for analyte gas 102 and buffer gas 105 may be separate gas supplies. In some embodiments, gas supplies 104 comprise one or more gas tanks for supplying analyte gas 103 and buffer gas 105. In some embodiments, gas supplies 104 comprise means for directly connecting a source of gas (e.g. a subject for exhaled breath analysis, a gas exhaust pipe, etc.) to gas cell 102. For example, in exhaled breath applications, gas supplies 104 may comprise tubes, connectors, respirators and/or other devices for connecting a subject to gas cell 102 for collecting the subject's exhaled breath and directly feeding the collected breath into gas cell 102 as analyte gas 103.

Gas cell 102 may be of any suitable design such that one or more operating parameters of gas cell 102 can be monitored and controlled, including, for example, temperature, pressure, and/or the like. In some embodiments, system 100 comprises a temperature control unit for monitoring and controlling a temperature of the gas mixture within gas cell 102. In some embodiments, the temperature control unit comprises a temperature-regulated housing which houses gas cell 102. A temperature within gas cell 102 can be increased or decreased relative to a temperature of the ambient environment (e.g. outside of gas cell 102) by increasing or decreasing the temperature within the temperature-regulated housing. Increasing the temperature within gas cell 102 relative to a temperature of the ambient environment may increase the average speed of molecules of analyte gas 103 and buffer gas 105 in gas cell 102 compared to the average speed of the molecules at the temperature of the ambient environment (e.g. outside of gas cell 102).

In some embodiments, system 100 comprises a sub-system for monitoring and controlling the pressure in gas supplies 104 and/or gas cell 102. For example, the sub-system monitors the pressure of gas supplies 104 and gas cell 102 to generate and/or maintain a pressure differential between gas supplies 104 and gas cell 102 for directing analyte gas 103 and buffer gas 105 into gas cell 102. In some embodiments, the sub-system comprises one or more pressure control systems for monitoring and/or controlling the pressure in gas cell 102 and gas supplies 104.

In some embodiments, gas cell 102 comprises a fluid-impermeable structure for confining analyte gas 103 and buffer gas 105 within gas cell 102. In some embodiments, gas cell 102 is connected to gas supplies 104 in a sealed manner to prevent gas leakage and any unexpected pressure change in gas cell 102.

Gas cell 102 may comprise any suitable means for mixing analyte gas 103 and buffer gas 105. Analyte gas 103 and buffer gas 105 may be mixed in any suitable manner. In some embodiments, analyte gas 103 is collected from an analyte source and subsequently mixed with buffer gas 105. In some embodiments, buffer gas 105 is mixed with or arranged to interact with analyte gas 103 at the analyte source such that sample gas collected from the analyte source already has a mixture of analyte gas 103 and buffer gas 105. Additional buffer gas 105 may be optionally mixed with the collected sample gas subsequent to collection and prior to excitation.

In some embodiments, buffer gas 105 is mixed with analyte gas 103 at the analyte source in a controlled manner to substantially exclude gas molecules that may interfere with the gas analysis from being present in the gas mixture (e.g. carbon dioxide, Argon, Neon, Methane, Hydrogen, water vapor, etc.). For example, in exhaled breath applications, a specially constituted preparation gas that includes buffer gas 105 and oxygen may be supplied to a subject to inhale instead of ambient air. By substituting ambient air with the preparation gas (e.g. during inhalation or otherwise), gas molecules that may interfere with the gas analysis could be substantially excluded from the collected sample gas mixture. In some embodiments, the preparation gas comprises a mixture of oxygen and buffer gas 105. In some embodiments, the mole fraction of the oxygen in the preparation gas mixture is in a range of about 15% to about 25% and the mole fraction of buffer gas 105 in the preparation gas mixture is in a range of about 75% to about 85%. In some embodiments, the mole fraction of oxygen in the preparation gas mixture is in a range of about 20% to about 22% and the mole fraction of buffer gas 105 in the preparation gas mixture is about 80% to about 82% (e.g. 21% Oxygen and 79% Helium, or 21% Oxygen and 79% Nitrogen, etc.). The subject may breathe in the preparation gas for a duration in the range of 5 to 40 minutes before the sample gas mixture is collected from the subject.

Analyte gas 103 and buffer gas 105 may be mixed at any suitable partial pressures. In some embodiments, analyte gas 103 is supplied to gas cell 102 at a partial pressure in a range of about 1 psi to about 30 psi. In a non-limiting example embodiment, the partial pressure of analyte gas 103 is about 20 psi. In some embodiments, buffer gas 105 is supplied to gas cell 102 at a partial pressure in a range of about 20 psi to about 110 psi. In some embodiments, the partial pressure of buffer gas 105 is in a range of about 70 psi to about 80 psi.

Analyte gas 103 and buffer gas 105 may be of any suitable concentration in the gas mixture. In some embodiments, analyte gas 103 has a mole fraction in a range of about 1% to about 80%. In a non-limiting example embodiment, analyte gas 103 has a mole fraction of about 20%. In some embodiments, buffer gas 105 has a mole fraction in a range of about 20% to about 99%. In a non-limiting example embodiment, the mole fraction of buffer gas 105 is about 80%.

System 100 comprises an optical excitation source 106 optically connected (indicated by dotted arrow in FIG. 1) to gas cell 102 for providing an optical excitation beam 107 into gas cell 102. Optical excitation beam 107 is selected such that an interaction between photons of optical excitation beam 107 and molecules of analyte gas 103 may result in Raman scattering (i.e. resulting in the emission of Raman scattered photons 109). Optical excitation beam 107 may have any suitable optical properties (including, but are not limited to, wavelength(s), power, bandwidth, pulse energy, pulse duration, pulse repetition rate, etc.) for generating Raman scattering with analyte gas 103. In some embodiments, optical excitation beam 107 comprises a pulsed optical excitation beam. Pulsed optical excitation beam may generate stimulated Raman scattering above a threshold pump power of the pulsed optical excitation beam. In some embodiments, optical excitation beam 107 has a pulse duration in a range of about 0.01 ns to about 15 ns. In some embodiments, optical excitation beam 107 has a pulse repetition rate in a range of about 0.1 Hz to about 100 MHz. In some embodiments, optical excitation beam 107 has a spectral bandwidth of less than 20 cm⁻¹. In some embodiments, optical excitation source 106 comprises a laser excitation source and optical excitation beam 107 comprises a laser excitation beam. Optical excitation source 106 may comprise any suitable optical elements for providing excitation beam 107 to gas cell 102.

Gas cell 102 may be of any suitable design and be made of any suitable material to facilitate interaction between light (excitation beam 107) and gas contained therein. In some embodiments, gas cell 102 is shaped to define a constricted cavity to promote interaction between light and gas. In some embodiments, gas cell 102 comprises a hollow-core optical fiber comprising a hollow core along a longitudinal extension of the fiber that permits permeation of gas within the hollow core.

In some embodiments, the hollow-core optical fiber comprises a band-gap transmission hollow-core fiber. A band-gap hollow-core fiber comprises micro-structured cladding features to reflect light of certain wavelengths, thereby preventing light of these wavelengths from escaping the hollow core. In some embodiments, the hollow-core fiber comprises a hollow-core photonic crystal fiber (HCPCF). In some embodiments, the hollow-core fiber comprises an anti-resonant hollow-core fiber. An anti-resonant hollow-core fiber comprises capillary walls in its micro-structured cladding features, such that light of specific wavelengths are anti-resonant to the thickness of the capillary walls and is not coupled into the cladding modes, thereby preventing light of these wavelengths from escaping the hollow core.

Optical excitation beam 107 may be substantially confined by gas cell 102 to interact with the gas mixture in gas cell 102 which includes analyte gas 103 and buffer gas 105 and to thereby scatter to generate Raman scattered photons 109. In some embodiments, gas cell 102 comprises a hollow-core fiber and optical excitation beam 107 is confined to a hollow core of the hollow-core fiber. The hollow-core fiber may facilitate generating stimulated Raman scattering resulting from an interaction between optical excitation beam 107 and the gas mixture within the hollow core. In the stimulated Raman scattering regime, a higher Raman intensity may result from the interaction between optical excitation beam 107 and the gas mixture in a relatively more constricted (relatively higher photon density) hollow core than from the interaction between optical excitation beam 107 and the gas mixture in a relatively less constricted (relatively lower photon density) hollow core. In some embodiments, the hollow core has a core diameter in a range of about 2 μm to about 50 μm. In some embodiments, the hollow-core fiber has a longitudinal length in a range of about 0.5 m to about 20 m.

Buffer gas 105 is selected such that the presence of buffer gas 105 may result in higher Raman intensity resulting from the interaction between optical excitation beam 107 and the gas mixture than from an interaction between optical excitation beam 107 and the gas mixture in the absence of buffer gas 105. The Raman intensity is proportional to or correlated with the number of Raman scattered photons 109 detected per unit time, for example, counts per second, for given wavenumbers or wavelengths.

The presence of buffer gas 105 may enhance Raman scattering by a variety of mechanisms. For example, without wishing to be bound by theory, one mechanism by which the presence of buffer gas 105 may enhance Raman scattering is that the presence of buffer gas 105 may increase the average interaction pathlength between photons of optical excitation beam 107 and molecules of analyte gas 103. When buffer gas 105 is mixed with analyte gas 103, optical excitation beam 107 may scatter off molecules of buffer gas 105 isotopically, which effectively increases the traveling distance of photons of optical excitation beam 107 compared to when buffer gas 105 is absent from the gas mixture. In other words, an average interaction pathlength between photons of excitation beam 107 and molecules of analyte gas 103 in the gas mixture may be greater than an average interaction pathlength between photons of excitation beam 107 and molecules of analyte gas 103 in an absence of buffer gas 105. The longer average interaction pathlength may provide more opportunity for photons of optical excitation beam 107 to interact inelastically with molecules of analyte gas 103.

Buffer gas 105 may comprise any suitable gas. In some embodiments, buffer gas 105 comprises an inert gas. In some embodiments, buffer gas 105 comprises a noble gas. In some embodiments, buffer gas 105 comprises Helium (He). In some embodiments, buffer gas 105 comprises Nitrogen gas (N₂).

System 100 comprises a detector 108 optically connected (indicated by dotted arrow in FIG. 1) to gas cell 102 for detecting Raman scattered photons 109 resulting from interaction between the photons of optical excitation beam 107 and the molecules of analyte gas 103 to thereby determine a spectral content 111 of Raman scattered photons 109. Spectral content 111 contains information about Raman intensity for given wavenumbers (or wavelengths). The Raman intensity of spectral content 111 is proportional to or correlated with the number of Raman scattered photons 109 detected per unit time, for example, counts per second, for a range of wavenumbers or wavelengths by detector 108 (referred to herein as “Raman intensity”). Spectral content 111 may be plotted to yield a Raman intensity curve over the range of wavenumbers or wavelengths. In some embodiments, calculating a value of the Raman intensity for a given wavenumber or wavelength comprises integrating the values of the Raman intensity curve (i.e. summing over the area under the Raman intensity curve) between a lower bound (i.e. a wavenumber or wavelength lower than the given wavenumber or wavelength) and an upper bound (i.e. a wavenumber or wavelength higher than the given wavenumber or wavelength). Any suitable lower and upper wavenumber/wavelength bounds may be selected for such integration, e.g., 3 dB points (as measured from the peak or from the average baseline), some threshold relative to average baseline and/or the like. The Raman intensity curve may contain wavenumbers or wavelengths having relatively higher Raman intensities compared to other wavenumbers or wavelengths, thus visually forming one or more Raman peaks in the Raman intensity curve. The Raman intensity of spectral content 111 may be quantified in normalized arbitrary units (a.u.). The normalized scale is useful for comparing intensities of different Raman peaks within the same spectrum or for comparing intensities of Raman peaks across spectra obtained under similar conditions. The normalized scale may be determined by any suitable process, including one or more steps of baseline correction, calibration for instruments, value normalization, error correction and/or the like. Detector 108 may comprise any suitable optical components for collecting Raman scattered photons 109 and any suitable signal processing hardware and/or software for generating spectral content 111 of the Raman scattered photons 109. In some embodiments, detector 108 comprises a spectrometer for detecting Raman scattered photons 109 and determining spectral content 111.

In some embodiments, optical excitation beam 107 is prevented from reaching detector 108. System 100 may comprise any suitable optical devices to block or otherwise prevent optical excitation beam from reaching detector 108.

System 100 comprises an analyzer 110 in communication (indicated by a dotted-and-dashed arrow in FIG. 1) with detector 108 for analyzing spectral content 111 to thereby determine the presence of at least one constituent of analyte gas 103. In some embodiments, determining the presence of at least one constituent of analyte gas 103 comprises identifying one or more wavenumbers (and/or wavelengths) in spectral content 111 that have relatively higher Raman intensity compared to other wavenumbers (and/or wavelengths) in the spectral content and, based on the identified one or more wavenumbers (and/or wavelengths) with relatively higher Raman intensity, determining an identity of the corresponding one or more constituents of analyte gas 103. In some embodiments, determining the presence of at least one constituent of analyte gas 103 is based on identifying at least one Raman peak in plotted spectral content 111 corresponding to the at least one constituent and the magnitude of the at least one Raman peak is greater than a magnitude of the at least one Raman peak in the absence of buffer gas 105. Analyzer 110 may comprise any suitable general purpose computer/processor(s) which may be suitable configured (e.g. with suitable software and/or hardware) for providing the functionality described herein.

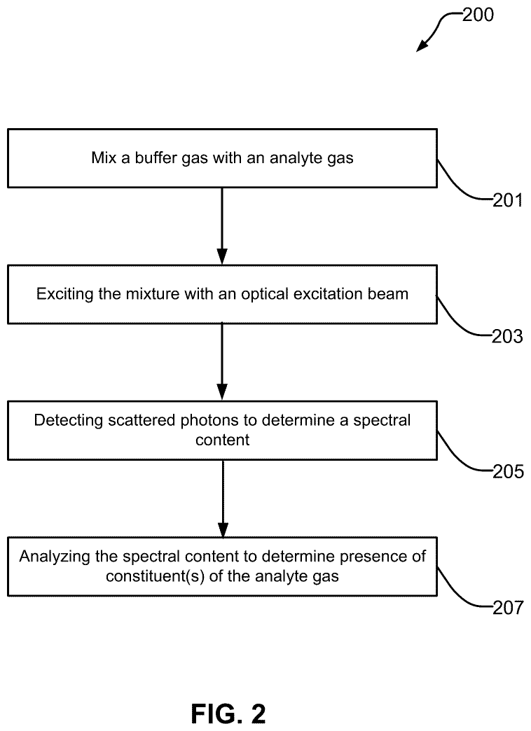

FIG. 2 is a flowchart of a method 200 for detecting a presence of at least one constituent of an analyte gas based on Raman scattering according to an example embodiment. Method 200 may be performed by any suitable systems disclosed herein, including, but not limited to, systems 100, 300, etc.

Method 200 begins with step 201 of mixing a buffer gas (e.g. buffer gas 105) with an analyte gas (e.g. analyte gas 103). The buffer gas and the analyte gas may be mixed by any suitable means in any suitable order. In some embodiments, the analyte gas is collected from a sample and then subsequently mixed with the buffer gas. In some embodiments, the buffer gas is mixed with or arranged to interact with the analyte gas at an analyte source and the gas mixture containing both the buffer gas and the analyte gas is directly collected from the analyte source. In some embodiments, the buffer gas is added to the analyte gas after the analyte gas is introduced into gas cell 102. The gas mixture may be contained in a gas cell (e.g. gas cell 102) for exposure to an optical excitation beam.

The buffer gas may be mixed with the analyte gas at any suitable partial pressures and/or in any suitable proportions. In some embodiments, the buffer gas has a buffer gas partial pressure and the analyte gas has an analyte gas partial pressure where the buffer gas partial pressure is different from the analyte gas partial pressure. In some embodiments, the buffer gas partial pressure is higher than the analyte gas partial pressure. In some embodiments, the buffer gas partial pressure is lower than the analyte gas partial pressure. In some embodiments, the buffer gas is supplied to the gas cell at the same partial pressure as the analyte gas. In some embodiments, a ratio of the buffer gas partial pressure to the analyte gas partial pressure is in a range of about 0.01 to about 1000.

In some embodiments, the analyte gas is supplied to a gas cell at a partial pressure in a range of about 1 psi to about 30 psi. In a non-limiting example embodiment, the partial pressure of the analyte gas is about 20 psi. In some embodiments, the buffer gas is supplied to the gas cell at a partial pressure in a range of about 20 psi to about 110 psi. In some embodiments, the partial pressure of the buffer gas is in a range of about 70 psi to about 80 psi. In some embodiments, the analyte gas has a mole fraction in a range of about 1% to about 80%. In a non-limiting example embodiment, the analyte gas has a mole fraction of about 20%. In some embodiments, the buffer gas has a mole fraction in a range of about 20% to about 99%. In a non-limiting example embodiment, the mole fraction of the buffer gas is about 80%.

In some embodiments, buffer gas is mixed with analyte gas at the analyte source in a controlled manner to substantially exclude gas molecules that may interfere with the gas analysis (contaminant molecules) from being present in the gas mixture (contaminant molecules may include, for example, carbon dioxide, Argon, Neon, Methane, Hydrogen, water vapor, etc.). For example, in exhaled breath applications, a specially constituted preparation gas that includes buffer gas and oxygen may be supplied to a subject to inhale and subsequently exhale instead of ambient air. By substituting ambient air with the preparation gas, gas molecules that may interfere with the gas analysis could be substantially excluded from the collected sample gas mixture. In some embodiments, the preparation gas comprises a mixture of oxygen and buffer gas 105. In some embodiments, the mole fraction of the oxygen in the preparation gas mixture is in a range of about 15% to about 25% and the mole fraction of buffer gas 105 in the preparation gas mixture is in a range of about 75% to about 85%. In some embodiments, the mole fraction of oxygen in the preparation gas mixture is in a range of about 20% to about 22% and the mole fraction of buffer gas 105 in the preparation gas mixture is about 80% to about 82% (e.g. 21% Oxygen and 79% Helium, or 21% Oxygen and 79% Nitrogen, etc.). The subject may breathe in the preparation gas for a duration in the range of 5 to 40 minutes before the sample gas mixture is collected from the subject.

In some embodiments, method 200 comprises the optional step of regulating a temperature of the gas mixture to promote mixing of the analyte gas and the buffer gas. In some embodiments, the temperature of the gas mixture is regulated to be maintained at a stable operating temperature. The operating temperature may be any temperature between about a temperature of the ambient environment and an upper threshold temperature. The upper threshold temperature may be set to a temperature above which hardware components of the system may malfunction. In some embodiments, the upper threshold temperature is about 50 degrees Celsius. In some embodiments, regulating the temperature of the gas mixture comprises increasing the temperature of the gas relative to a temperature of the ambient environment to thereby increase the average speed of the molecules of the gas mixture compared to an average speed of the molecules of the gas mixture at the temperature of the ambient environment.

Method 200 then proceeds with step 203 of exciting the gas mixture of the analyte gas and the buffer gas with an optical excitation beam (e.g. optical excitation beam 107). The optical excitation beam is selected such that Raman scattering may result from an interaction between photons of the optical excitation beam and molecules of the analyte gas. In some embodiments, the optical excitation beam comprises a laser excitation beam. The optical excitation beam may be subject to any suitable optical manipulation prior to interacting with the gas mixture. For example, the optical excitation beam may be filtered, focused, reflected, steered and/or the like in any suitable manner prior to the optical excitation beam interacting with the gas mixture.

The interaction between the optical excitation beam and the gas mixture may result in the emission of Raman scattered photons (e.g. Raman scattered photons 109). Step 205 of method 200 comprises detecting the Raman scattered photons resulting from the interaction between photons of the optical excitation beam and molecules of the gas mixture to thereby determine a spectral content (e.g. spectral content 111) of the Raman scattered photons. In some embodiments, the spectral content contains information about Raman intensity over a range of wavenumbers or wavelengths.

The Raman scattered photons may be detected by any suitable detector(s). In some embodiments, the Raman scattered photons are detected by a spectrometer. The Raman scattered photons may be optically manipulated in any suitable manner before being detected by a detector. For example, the Raman scattered photons may be filtered, focused, reflected, steered and/or the like prior to being detected by the detector. The Raman scattered photons may be directed to the detector by any suitable means. In some embodiments, the Raman scattered photons are collected by an optical system that comprises an optical fiber (e.g. a multi-mode fiber) and directed to the detector by the optical fiber.

Method 200 then proceeds to step 207 which involves analyzing the spectral content to determine the presence of at least one constituent of the analyte gas. In some embodiments, analyzing the spectral content to determine the presence of at least one constituent of the analyte gas comprises identifying one or more wavenumbers (or wavelengths) in the spectral content that have a corresponding relatively higher Raman intensity compared to other wavenumbers (or wavelengths) in the spectral content and, based on the identified one or more wavenumbers (or wavelengths), determining an identity of the corresponding one or more constituents of the analyte gas. In some embodiments, determining the presence of at least one constituent of the analyte gas is based on identifying at least one Raman peak in the plotted spectral content corresponding to the at least one constituent and the magnitude of the at least one Raman peak is greater than a magnitude of the at least one Raman peak in the absence of the buffer gas. The analysis may be performed by any suitable analyzer. In some embodiments, the analyzer comprises general purpose computer/processor(s) which may be suitable configured (e.g. with suitable software and/or hardware) for providing the functionality described herein.

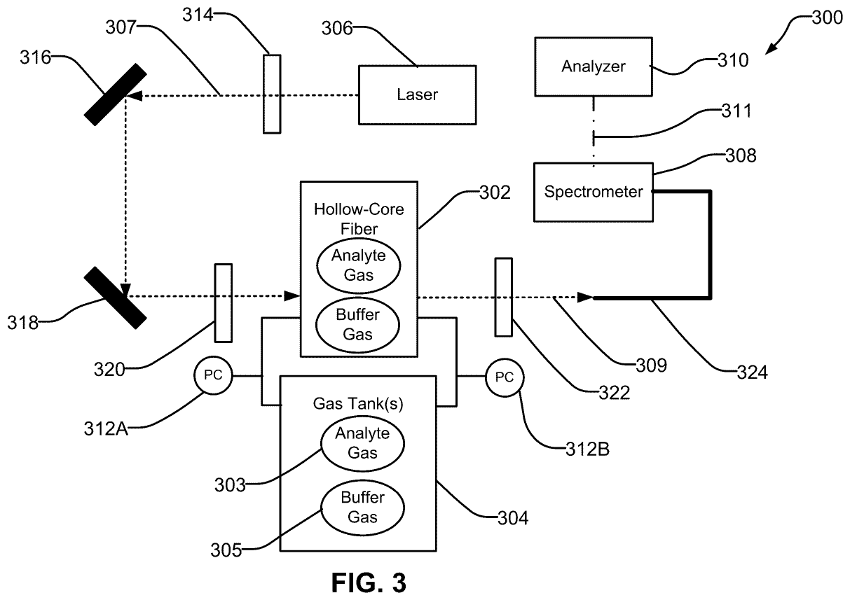

FIG. 3 is a schematic diagram of a system 300 for gas analysis based on Raman scattering according to another example embodiment. System 300 may be used to determine a presence of at least one constituent of an analyte gas, for example, by applying method 200. System 300 is similar to system 100. Features of system 300 that are similar to corresponding features of system 100 are labelled with the same reference numerals except that those features of system 300 are labelled in 300-series and the corresponding features of system 100 are labelled in 100-series.

System 300 comprises one or more gas tanks 304 in fluid connection with a hollow-core optical fiber 302 for supplying an analyte gas 303 and a buffer gas 305 to hollow-core optical fiber 302. Hollow-core optical fiber 302 is shaped to define a hollow core along a longitudinal extension of the fiber wherein the hollow core permits permeation of fluid (e.g. gas) within the hollow core.

In some embodiments, hollow-core optical fiber 302 comprises a band-gap transmission hollow-core fiber. In some embodiments, the hollow-core fiber comprises an anti-resonant hollow-core fiber. In some embodiments, the hollow-core fiber comprises a hollow-core photonic crystal fiber (HCPCF). In some embodiments, the hollow core has a core diameter in a range of about 2 μm to about 50 μm. In some embodiments, the hollow-core fiber has a longitudinal length in a range of about 0.5 m to about 20 m.

System 300 comprises pressure control systems 312A and 312B in fluid communication with gas tank 304 and hollow-core fiber 302 for monitoring and/or controlling the pressure in gas tanks 304 and hollow-core optical fiber 302. For example, pressure control systems 312A and 312B may control the pressure of gas tanks 304 and hollow-core optical fiber 302 to generate and/or maintain a pressure differential between gas tanks 304 and hollow-core optical fiber 302 for directing analyte gas 303 and buffer gas 305 into the hollow core of hollow-core optical fiber 302.

In some embodiments, pressure control system 312A is configured to supply one of analyte gas 303 and buffer gas 305 to hollow-core fiber 302 at a first partial pressure and pressure control system 312B is configured to supply the other one of analyte gas 303 and buffer gas 305 at a second partial pressure. In some embodiments, the first partial pressure is different from the second partial pressure. In some embodiments, the first pressure is the same as the second partial pressure. In some embodiments, the partial pressure of buffer gas 305 is higher than the partial pressure of analyte gas 303. Hollow-core optical fiber 302 may be connected to gas tanks 304 in a sealed manner to prevent leakage of analyte gas 303 and buffer gas 305 as well as any unexpected drop in pressure in gas tanks 304 and/or hollow-core optical fiber 302.

System 300 comprises a laser source 306 optically connected to hollow-core fiber 302 for emitting a laser excitation light 307 into the hollow core of hollow-core fiber 302. Laser source 306 may comprise any suitable lasers. In some embodiments, laser source 306 comprises a continuous wave (CW) laser. In some embodiments, laser source 306 comprises a pulsed laser configured to emit pulsed laser excitation beams. In some embodiments, laser source 306 comprises an optical parametric oscillator (OPO)-based laser. In some embodiments, laser source 306 comprises a dye laser.

Laser excitation light 307 may have any suitable optical properties (including, but are not limited to, wavelength(s), power, bandwidth, pulse energy, pulse duration, pulse repetition rate, etc.) for generating Raman scattering with analyte gas 303. In some embodiments, laser excitation beam 307 has a pulse duration in a range of about 0.01 ns to about 15 ns. In some embodiments, laser excitation beam 307 has a pulse repetition rate in a range of about 0.1 Hz to about 100 MHz. In some embodiments, laser excitation beam 107 has a spectral bandwidth of less than 20 cm⁻¹. In a non-limiting example embodiment, laser excitation light 307 is tuned to a wavelength of 785 nm with a pulse duration of 7 ns, a pulse repetition rate of 20 Hz and a bandwidth of 4-7 cm⁻¹. In another non-limiting example embodiment, laser excitation light 307 is has a pulse duration of 0.8 ns, with a pulse repetition rate of 7 Hz and a spectral bandwidth of 0.7 cm⁻¹. Pulsed laser excitation beam 307 may generate stimulated Raman scattering above a threshold pump power of pulsed laser excitation beam 307. Hollow-core fiber 302 may facilitate generating stimulated Raman scattering resulting from an interaction between laser excitation beam 307 and the gas mixture within the hollow core. In the stimulated Raman scattering regime, a higher Raman intensity may result from the interaction between laser excitation beam 307 and the gas mixture in a relatively more constricted hollow core (which provides relative high photon density) than from the interaction between laser excitation beam 307 and the gas mixture in a relatively less constricted hollow core (which provides relatively low photon density).

System 300 may comprise any suitable optical elements located between laser source 306 and hollow-core fiber 302 for directing and/or otherwise manipulating laser excitation light 307 before laser excitation light 307 is coupled into the hollow core of hollow-core fiber 302. In some embodiments, system 300 comprises optical elements 314 for filtering laser excitation light 307. In some embodiments, optical elements 314 comprises one or more of: laser line filter, neutral density filter, or any other suitable optical filters for filtering laser excitation light 307. In some embodiments, system 300 comprises mirrors/reflectors 316 and 318 for optically aligning laser excitation light 307 with hollow-core fiber 302. Mirrors/reflectors 316 and 318 may comprise any suitable optical devices for directing laser light. In some embodiments, system 300 comprises optical element(s) 320 for focusing laser excitation light 307 into the hollow core of hollow-core fiber 302. In some embodiments, optical element(s) 320 comprises an aspheric lens.

Laser excitation beam 307 may be substantially confined by hollow-core fiber 302 to interact with the gas mixture in hollow-core fiber 302 which includes analyte gas 303 and buffer gas 305 and to thereby scatter to generate Raman scattered photons. For example, a band-gap hollow-core fiber comprises micro-structured cladding features to reflect laser light of certain wavelengths, thereby preventing laser light of these wavelengths from escaping the hollow core. As another example, an anti-resonant hollow-core fiber comprises capillary walls in its micro-structured cladding features, such that light of specific wavelengths are anti-resonant to the thickness of the capillary walls and is not coupled into the cladding modes, thereby preventing laser light of these wavelengths from escaping the hollow core.

Buffer gas 305 may be selected such that the presence of buffer gas 305 results in higher Raman intensity resulting from the interaction between laser excitation beam 307 and the gas mixture than from the interaction between laser excitation beam 307 and the gas mixture in the absence of buffer gas 305. For example, the presence of buffer gas 305 may increase the average interaction pathlength between photons of laser excitation beam 307 and molecules of analyte gas 303. In other words, an average interaction pathlength between photons of laser excitation beam 307 and molecules of analyte gas 303 in the gas mixture (including analyte gas 303 and buffer gas 305) is greater than an average interaction pathlength between photons of laser excitation beam 307 and molecules of analyte gas 303 in an absence of buffer gas 305.

System 300 comprises a spectrometer 308 optically connected to hollow-core fiber 302 for detecting Raman scattered photons 309 resulting from interaction between photons of laser excitation beam 307 and molecules of analyte gas 303 to thereby determine a spectral content 311 of Raman scattered photons 309. Spectral content 311 is similar to spectral content 111 and contains information about Raman intensity for a range of wavenumbers or wavelengths.

System 300 may comprise any suitable optical elements located between hollow-core optical fiber 302 and spectrometer 308 for collecting, filtering, directing and/or the like Raman scattered photons 309 to spectrometer 308. In some embodiments, system 300 comprises optical element(s) 322 for collimating the scattered photons. In some embodiments, optical element(s) 322 comprise a lens. In some embodiments, optical element(s) 322 comprise a wavelength selective filter for blocking laser excitation light 307 (i.e. laser light having the wavelength of the incident laser excitation light 307).

System 300 may comprise any suitable medium for directing Raman scattered photons 309 to spectrometer 308. In some embodiments, system 300 comprises an optical fiber 324 for collecting Raman scattered photons 309 and directing Raman scattered photons 309 to spectrometer 308. In some embodiments, optical fiber 324 comprises a multi-mode fiber. In some embodiments, optical element 322 comprises one or more additional lenses or other optical elements for focusing Raman scattered photons 309 onto optical fiber 324.

System 300 comprises an analyzer 310 in communication with spectrometer 308 for analyzing spectral content 311 to thereby determine the presence of at least one constituent of analyte gas 303. In some embodiments, determining the presence of at least one constituent of analyte gas 303 comprises identifying one or more wavenumbers (or wavelengths) in spectral content 311 that have relatively higher Raman intensity compared to other wavenumbers (or wavelengths) in the spectral content and, based on the identified one or more wavenumbers (or wavelengths) with higher Raman intensity, determining an identity of the corresponding one or more constituents of analyte gas 303. Analyzer 310 may comprise any suitable general purpose computer/processor(s) which may be suitable configured (e.g. with suitable software and/or hardware) for providing the functionality described herein.

In other respects, system 300 may be similar to those of system 100 and/or any of the other systems described herein.

An aspect of the invention relates to systems and methods for gas analysis based on pulsed single-beam stimulated Raman scattering (SRS) and fiber-enhanced Raman scattering (FERS).

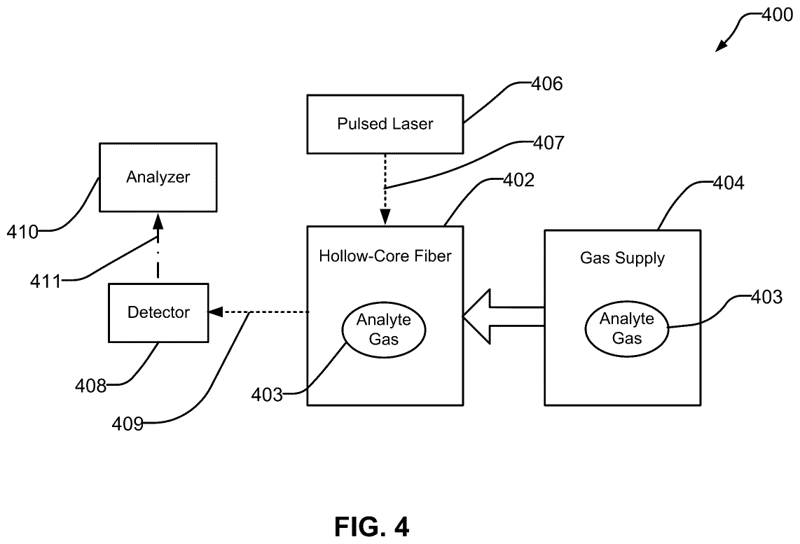

FIG. 4 is a schematic diagram of a system 400 for gas analysis based on single-beam SRS FERS according to an example embodiment. System 400 may be applied to determine a presence of at least one constituent of an analyte gas 403. System 400 is similar to system 100, except that analyte gas supply 404 is not required to supply buffer gas to hollow-core fiber 402 (i.e. the use of buffer gas is optional in system 400 and not illustrated in FIG. 4) and that system 400 comprises a pulsed laser 406 for excitation. Components of system 400 similar to components of system 100 are labelled with the same reference numerals except that these components of system 400 are labeled in 400-series and the corresponding components of system 100 are labeled in 100-series.

System 400 comprises an analyte gas supply 404 in fluid connection with a hollow-core fiber 402 for supplying an analyte gas 403 to hollow-core fiber 402. The hollow core of hollow-core fiber 402 permits the permeation of gas (e.g. analyte gas 403) within the hollow core and comprises micro-structures that substantially confine photons of pulsed laser excitation beam 407 to the hollow core to thereby facilitate interaction between photons of pulsed laser excitation beam 407 and molecules of analyte gas 403.

System 400 comprises a single-beam pulsed laser 406 optically connected to hollow-core fiber 402 for emitting a pulsed laser excitation beam 407 into the hollow core of hollow-core fiber 402. Single-beam pulsed laser 406 may comprise any suitable lasers for generating Raman scattering with analyte gas 403. In some embodiments, single-beam pulsed laser 406 comprises a dye laser. In some embodiments, single-beam pulsed laser 406 comprises an optical parametric oscillator (OPO) laser.

Pulsed laser excitation beam 407 may have any suitable optical properties (including, but not limited to, wavelength(s), power, bandwidth, pulse energy, pulse duration, pulse repetition rate, etc.) for generating Raman scattering with analyte gas 403. In some embodiments, pulsed laser excitation beam 407 has a pulse duration in a range of about 0.01 ns to about 15 ns. In some embodiments, pulsed laser excitation beam 407 has a pulse repetition rate in a range of about 0.1 Hz to about 100 MHz. In some embodiments, pulsed laser excitation beam 407 has a spectral bandwidth of less than 20 cm⁻¹. In a non-limiting example embodiment, pulsed laser excitation beam 407 is tuned to a wavelength of 785 nm with a pulse duration of 7 ns, a pulse repetition rate of 20 Hz and a bandwidth of 4-7 cm⁻¹. In another non-limiting example embodiment, pulsed laser excitation beam 407 is has a pulse duration of 0.8 ns, with a pulse repetition rate of 7 Hz and a spectral bandwidth of 0.7 cm−¹. System 400 may comprise any suitable optical elements located in an optical path between single-beam pulsed laser 406 and hollow-core fiber 402 to optically manipulate pulsed laser excitation beam 407.

Pulsed laser excitation beam 407 is applied to analyte gas 403 above a threshold laser pump power for generating stimulated Raman scattering. The threshold laser pump power for generating stimulated Raman scattering depends, at least in part, on properties of analyte gas 403. In the stimulated Raman scattering regime (i.e. when pulsed laser excitation beam 407 has a laser pump power above the threshold laser pump power), Raman intensity may increase exponentially with increase in the laser pump power.

System 400 comprises a detector 408 optically connected to hollow-core fiber 402 for generating a spectral content 411 based on detected Raman scattered photons 409. Detector 408 may be similar to detectors (e.g. detectors 108, 308) described elsewhere herein. Spectral content 411 is similar to spectral content (e.g. spectral content 111, 311) described elsewhere herein.

System 400 comprises an analyzer 410 in communication with detector 408 for receiving a spectral content 411 and thereby determining the presence of at least one constituent(s) of analyte gas 403 based on spectral content 411. Analyzer 410 may be similar to analyzers (e.g. analyzers 110, 310) described elsewhere herein.

In other respects, system 400 may be similar to those of system 100 and/or any of the other systems described herein.

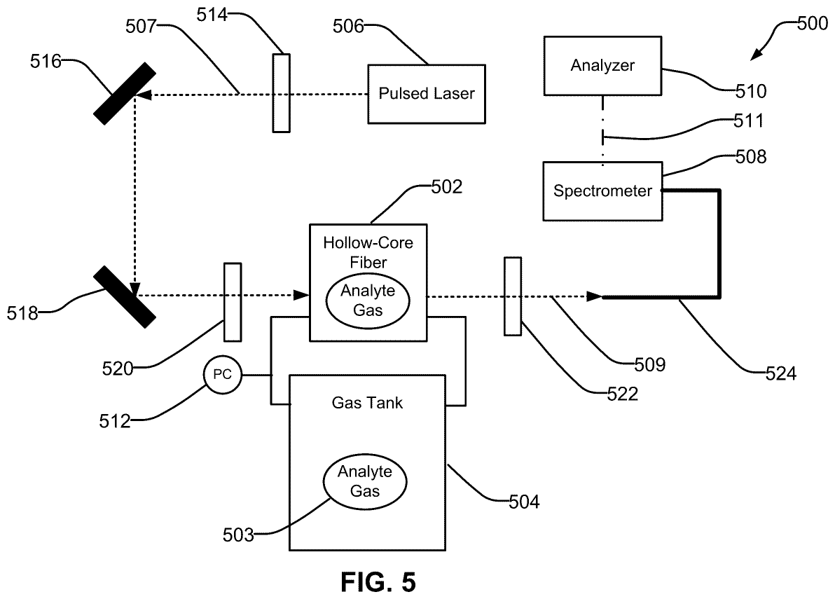

FIG. 5 is a schematic diagram of a system 500 for gas analysis based on single-beam SRS FERS according to another example embodiment. System 500 may be applied to determine a presence of at least one constituent of an analyte gas 503. System 500 is similar to system 400. System 500 is also similar to system 300, except that gas tank 504 is not required to supply buffer gas to hollow-core fiber 502 (i.e. the use of buffer gas is optional in system 500 and not illustrated in FIG. 5) and that system 500 comprises a pulsed laser 506. Components of system 500 similar to components of systems 300 or 400 are labelled with the same reference numerals except that these components of system 500 are labeled in 500-series and the corresponding components of systems 300 or 400 are labeled in 300-series or 400-series.

System 500 comprises a gas tank 504 in fluid connection with a hollow-core fiber 502 for supplying an analyte gas 503 to hollow-core fiber 502. The hollow core of hollow-core fiber 502 permits the permeation of gas (e.g. analyte gas 503) within the hollow core and comprises micro-structures that substantially confine pulsed laser excitation beam 507 to the hollow core to thereby facilitate interaction between photons of pulsed laser excitation beam 507 and molecules of analyte gas 503. Gas tank 504 is similar to gas tank 404.

System 500 comprises a pressure control system 512for monitoring and/or controlling the pressure of gas within hollow-core fiber 502. Pressure control system 512 of system 500 is similar to pressure control systems 312A and 312B of system 300.

System 500 comprises a single-beam pulsed laser 506 optically connected to hollow-core fiber 502 for emitting a pulsed laser excitation beam 507 into the hollow core of hollow-core fiber 502. Single-beam pulsed laser 506 and pulsed laser excitation beam 507 of system 500 may be similar to single-beam pulsed laser 406 and pulsed laser excitation beam 407 of system 400.

System 500 comprises optical elements 514, 516, 518 and 520 located between pulsed laser 506 and hollow-core fiber 502 for optically manipulating laser excitation beam 507 in any suitable manner. Optical elements 514, 516, 518 and 520 may be similar to optical elements 314, 316, 318 and 320 of system 300.

Pulsed laser excitation beam 507 is applied to analyte gas 503 above a threshold laser pump power for generating stimulated Raman scattering. The threshold laser pump power for generating stimulated Raman scattering depends, at least in part, on properties of analyte gas 503. In the stimulated Raman scattering regime (i.e. when pulsed laser excitation beam 507 has a laser pump power above the threshold laser pump power), Raman intensity may increase exponentially with increase in the laser pump power.

System 500 comprises a spectrometer 508 optically connected to hollow-core fiber 502 for generating a spectral content 511 based on detected Raman scattered photons 509. Spectrometer 508 may be similar to detectors 108, 308 and 408 described elsewhere herein. Spectral content 511 may be similar to spectral content 111, 311 and 411 described elsewhere herein. System 500 comprises optical elements 522 located between hollow-core fiber 502 and spectrometer 508 to apply any suitable optical manipulation to the scattered light. Optical elements 522 may be similar to optical elements 322. System 500 comprises an optical fiber 524 for collecting Raman scattered photons 509 and directing Raman scattered photons 509 to spectrometer 508. Optical fiber 524 may be similar to optical fiber 324.

System 500 comprises an analyzer 510 in communication with detector 508 for receiving a spectral content 511 and thereby determining the presence of at least one constituent(s) of analyte gas 503 based on spectral content 511. Analyzer 510 may be similar to analyzers 110, 310 and 410 described elsewhere herein.

In other respects, system 500 may be similar to those of system 100 and/or any of the other systems described herein.

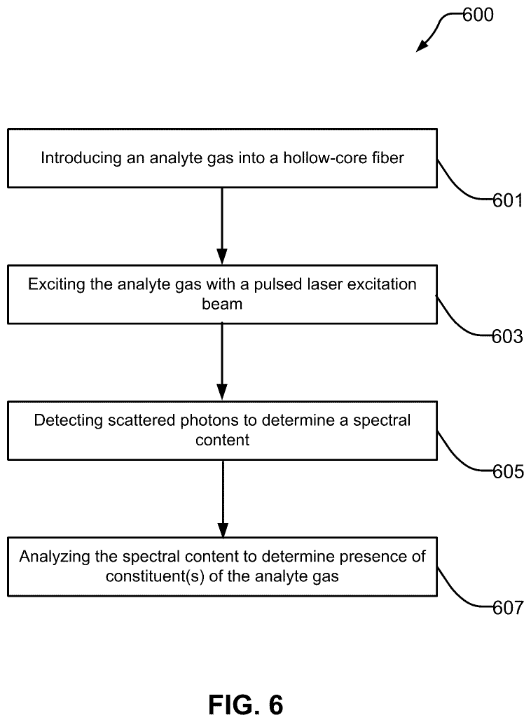

FIG. 6 is a flowchart of a method 600 for detecting a presence of at least one constituent of an analyte gas based on Raman scattering according to an example embodiment. Method 600 may be performed by any suitable systems disclosed herein, including, but are not limited to, systems 400, 500, etc.

Method 600 begins with step 601 of introducing an analyte gas (e.g. analyte gas 403, 503) into a hollow core of a hollow-core fiber (e.g. hollow-core 402, 502). The hollow core of the hollow-core fiber permits permeation of fluid within the hollow core. The analyte gas may be introduced into the hollow core of the hollow-core fiber at any suitable pressure. In some embodiments, the pressure of the analyte gas in the hollow-core fiber is in a range of about 20 psi to about 120 psi.

Method 600 proceeds with step 603 of exciting the analyte gas with a pulsed laser excitation beam (e.g. pulsed laser excitation beam 407, 507) above a threshold laser pump power to generate stimulated Raman scattering. The threshold laser pump power for generating stimulated Raman scattering depends, at least in part, on properties of the analyte gas. In the stimulated Raman scattering regime (i.e. when the pulsed laser excitation beam has a laser pump power above the threshold laser pump power), Raman intensity may increase exponentially with increase in the laser pump power.

Method 600 then proceeds with step 605 which involves detecting the Raman scattered photons (e.g. Raman scattered photons 409, 509) as a result of the interaction between photons of the pulsed laser excitation beam and molecules of the analyte gas (i.e. stimulated Raman scattering) to thereby determine a spectral content of the detected Raman scattered photons (e.g. spectral content 411, 511). In some embodiments, the spectral content contains information about Raman intensity for corresponding wavenumbers.

Method 600 then proceeds to step 607 of analyzing the spectral content of the detected Raman scattered photons to thereby determine the presence of at least one constituent of the analyte gas. In some embodiments, analyzing the spectral content to determine the presence of at least one constituent of the analyte gas comprises identifying one or more wavenumbers (or wavelengths) in the spectral content that have a corresponding relatively higher Raman intensity compared to other wavenumbers (or wavelengths) in the spectral content and, based on the identified one or more wavenumbers (or wavelengths), determining an identity of the corresponding one or more constituents of the analyte gas. The analysis may be carried out by suitable analyzer. In some embodiments, the analyzer comprises general purpose computer/processor(s) which may be suitable configured (e.g. with suitable software and/or hardware) for providing the functionality described herein.

In other respects, system 500 may be similar to those of system 400 and/or any of the other systems described herein. Experiments were performed to test the efficacy of the systems and methods disclosed herein.

A first set of experiments were performed to assess the performance of CERS based systems. For the experiments, the excitation pump beam is generated from pumping an Oxazine 750 Perchlorate dye laser with a 337 nm nitrogen laser with a pulse duration of 0.8 ns, a pulse repetition rate of 7 Hz and a spectral bandwidth of 0.04 nm (0.7 cm⁻¹ ). The output of the dye laser was tuned to 785 nm for Raman excitation. A 2 m long HCPCF was used as the gas cell. The HCPCF has a core diameter of 7.5±1 μm. The optimal transmission window of the HCPCF was from 770 nm to 870 nm, which allowed the excitation beam and the generated Stokes Raman photons to be transmitted in the fiber with minimal attenuation losses. Raman photons were collected in forward-scattered mode and were transferred to a spectrometer using a 50 μm multimode fiber for analysis. The pressure of the system was monitored and controlled using two commercial pressure control systems with an accuracy of ±1% in pressure reading.

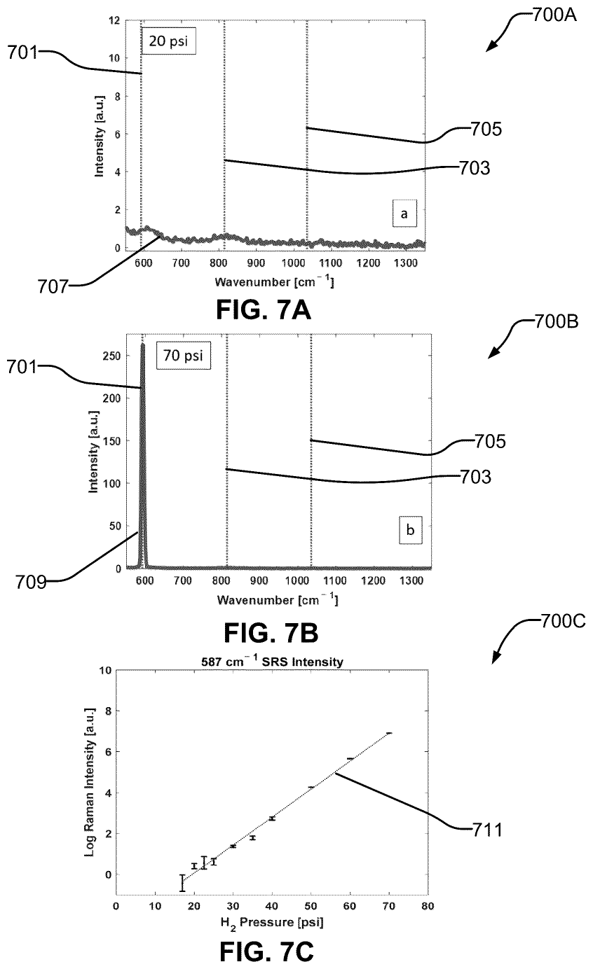

FIGS. 7A-C show the relationship between Raman intensity in normalized arbitrary units (a.u.) of an analyte gas (hydrogen gas (H₂) in this instance) and pressure of the analyte gas in a single-beam SRS FERS set-up and in absence of any buffer gas. Pressure of H2 was varied from about 17 psi to about 70 psi. The results serve as the baseline for comparison to measurements of CERS based systems. FIG. 7A is a plot 700A showing the Raman intensities of H₂ at 20 psi. FIG. 7B is a plot 700B showing the Raman intensities of H₂ over a range of wavenumbers at 70 psi. In both FIGS. 7A and 7B, the wavenumbers of the known Raman transitions of H₂ are denoted by vertical lines 701, 703 and 705. FIG. 7C is a plot 700C showing the relationship between the intensity of the stimulated Raman peak and pressure of H₂.

In FIG. 7A, the Raman intensity 707 is plotted against wavenumbers from about 550 cm⁻¹ to about 1300 cm⁻¹. As can be seen in plot 700A, the Raman intensities are not prominent at any of the known Raman transitions 701, 703 and 705. In FIG. 7B, the Raman intensity 709 is plotted against wavenumbers from about 550 cm⁻¹ to about 1300 cm⁻¹. As can be seen in plot 700B, The Raman intensity at known transition 701 (corresponding to a wavenumber of 587 cm⁻¹) has an intensity value of over 250 a.u. compared to the intensity value of about 1 a.u. in plot 700A. Plot 700C shows that there is a linear relationship between the log value of Raman intensity at wavenumber of 587 cm⁻¹ and the pressure of H₂ as shown by fitted line 711. In other words, Raman intensity increases exponentially per unit increase in total pressure of the analyte gas.

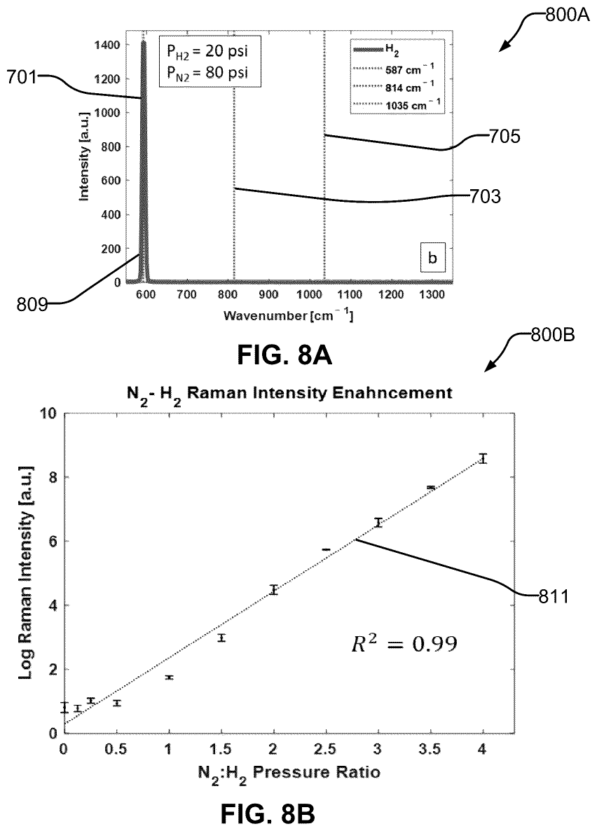

FIGS. 8A and 8B show the relationship between Raman intensity (a.u.) of an analyte gas (hydrogen gas H₂ in this instance) and the pressure ratio of a buffer gas (N₂ in FIGS. 8A and 8B) to the analyte gas in a CERS set-up. N₂ was mixed with H₂ at various partial pressures to generate the data presented in FIGS. 8A and 8B. FIG. 8A is a plot 800A showing the Raman intensities 809 of H₂ when H₂ has a partial pressure of 20 psi and N₂ has a partial pressure of 80 psi. In FIG. 8A, the wavenumbers of the known Raman transitions of H₂ are denoted by vertical lines 701, 703 and 705. FIG. 8B is a plot 800B showing the relationship between the log of the intensity of the stimulated Raman peak and the pressure ratio of N₂:H₂ ranging from 0 to 4.

As can be seen in FIG. 8A, the Raman intensity of known transition 701 (corresponding to a wavenumber of 587 cm⁻¹) has an intensity value of about 1400 a.u. compared to about 250 a.u. of plot 700B and about 1 a.u. of plot 700A. In FIG. 8B, plot 800B shows that there is a linear relationship between the log of the Raman intensity and the pressure ratio between N₂ and H₂ as shown by fitted line 811. In other words, Raman intensity increases exponentially with per unit increase in the partial pressure of the buffer gas N₂.

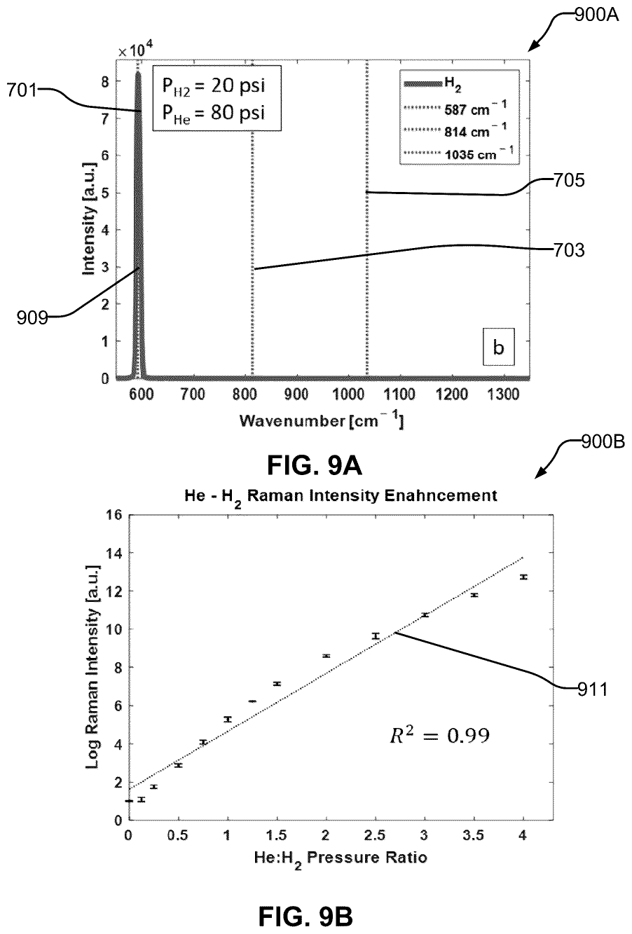

FIGS. 9A and 9B show the relationship between Raman intensity (a.u.) of an analyte gas (hydrogen gas H2 in this instance) and the pressure ratio of another buffer gas Helium (He in FIGS. 9A and 9B) to the analyte gas in a CERS set-up. He was mixed with H₂ at various partial pressures to generate the data presented in FIGS. 9A and 9B. FIG. 9A is a plot 900A showing the Raman intensities 909 of H₂ when H₂ has a partial pressure of 20 psi and He has a partial pressure of 80 psi. In FIG. 9A, the wavenumbers of the known Raman transitions of H₂ are denoted by vertical lines 701, 703 and 705. FIG. 9B is a plot 900B showing the relationship between the log of the intensity of the stimulated Raman peak and the pressure ratio of He:H₂ ranging from 0 to 4.

As can be seen in FIG. 9A, the Raman intensity of known transition 701 (corresponding to a wavenumber of 587 cm⁻¹) has an intensity value of about 8×10⁴ a.u. compared to about 1400 a.u. of plot 800A, about 250 a.u. of plot 700B and about 1 a.u. of plot 700A. In FIG. 9B, plot 900B shows that there is a linear relationship between the log of the Raman intensity and the pressure ratio between He and H₂ as shown by fitted line 911. In other words, Raman intensity increases exponentially with per unit increase in the partial pressure of the buffer gas He.

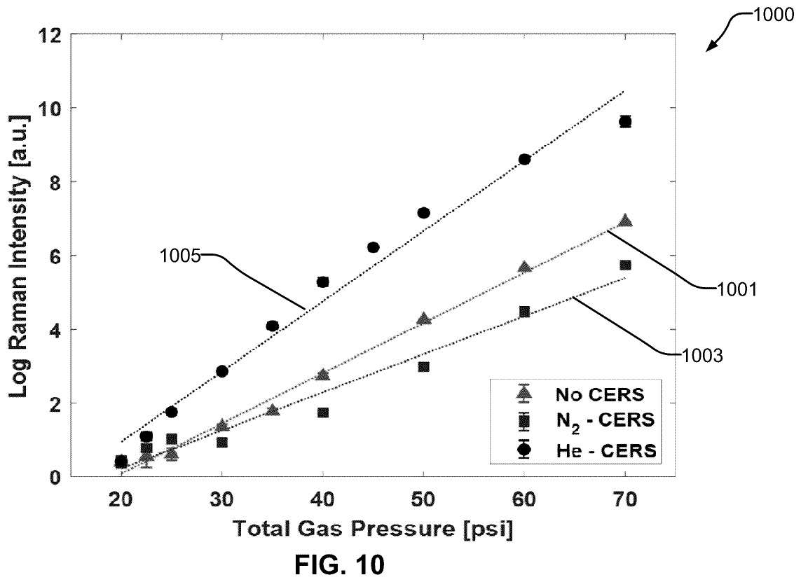

FIG. 10 is a plot 1000 comparing the rate of exponential increase (plotted in the log of Raman intensity) versus total gas pressure in three different setups:
No CERS (i.e. results shown in FIGS. 7A-C and indicated by triangles in FIG. 10) as shown by fitted line 1001;
N₂-CERS (i.e. results shown in FIGS. 8A and 8B and indicated by rectangles in FIG. 10) as shown by fitted line 1003; and,
He-CERS (i.e. results shown in FIGS. 9A and 9B and indicated by circles in FIG. 10) as shown by fitted line 1005.

The total gas pressure ranges from about 20 psi to about 70 psi. As can be seen from FIG. 10, He-CERS outperforms no CERS and N₂-CERS at almost all pressure levels. In other words, it is more efficient to mix He with an analyte gas and to increase the concentration of He rather than increasing the concentration of the analyte gas itself. Moreover, it may not be practical to increase the pressure of the analyte gas and/or the concentration of the constituents of interest in some applications. On the other hand, it is relatively easier to add a buffer gas such as He or N₂ and increase the partial pressure/concentration of the buffer gas. Therefore, CERS based systems provide a cost-effective and simple-to-implement Raman enhancement solution to gas analysis.

To assess the performance of CERS based systems in more practical settings, an experiment on a VOC was performed. Propene was chosen as the VOC to be tested with the CERS based system since propene was one of the many VOCs found in human breath and was readily available. Raman measurements were first obtained from pure propene at a pressure of 4 psi without any buffer gas. The pressure of 4 psi is close to the vapor pressure of some VOCs found in human breath. Raman spectra were then measured by adding He to the CERS system with increasing pressures of 20, 50, 80 and 110 psi. Propene partial pressure was held constant at 4 psi as He was introduced. The mole fraction of propene decreased to 16.7%, 7.4%, 4.8% and 3.5% respectively for 20, 50, 80 and 110 psi of added He.

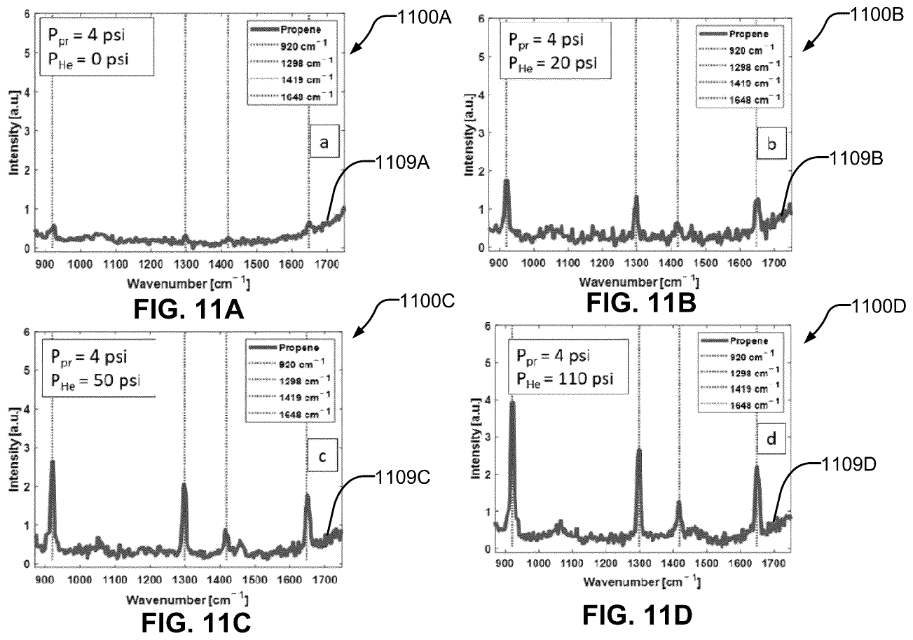

FIGS. 11A-D are plots 1100A-D showing the respective Raman intensities (a.u.) 1109A-D of propene when the partial pressure of the buffer gas helium is at 0 psi, 20 psi, 50 psi and 110 psi respectively. The wavenumbers of known propene Raman transitions, indicated by dotted vertical lines in plots 1100A-D, are 920 cm⁻¹, 1298 cm⁻¹, 1419 cm⁻¹ and 1648 cm⁻¹. As can be seen from FIGS. 11A-D, the Raman intensities at the wavenumbers of the known Raman transitions of propene increase as the partial pressure of the buffer gas helium increases. For example, the Raman intensity at 920 cm⁻¹ increased from about 0.5 a.u. when there is no helium to about 1.8 a.u. at 20 psi of helium, about 2.7 a.u. at 50 psi of helium and about 4 a.u. at 110 psi of helium. The enhancement of Raman intensity is meaningful with an increase of about 5x, 7x, 10x and 12x for helium at partial pressures of 20 psi, 50 psi, 80 psi and 110 psi respectively.

Therefore, the experiment on propene confirm that CERS systems are capable of detecting trace amount of minor components in a gas mixture under the right conditions and that the enhancement of Raman intensity with the addition of buffer gas is practically meaningful.

Where a component (e.g. a software module, processor, assembly, device, circuit, etc.) is referred to herein, unless otherwise indicated, reference to that component (including a reference to a “means”) should be interpreted as including as equivalents of that component any component which performs the function of the described component (i.e., that is functionally equivalent), including components which are not structurally equivalent to the disclosed structure which performs the function in the illustrated exemplary embodiments of the invention.

Embodiments of the invention may be implemented using specifically designed hardware, configurable hardware, programmable data processors configured by the provision of software (which may optionally comprise “firmware”) capable of executing on the data processors, special purpose computers or data processors that are specifically programmed, configured, or constructed to perform one or more steps in a method as explained in detail herein and/or combinations of two or more of these. Examples of specifically designed hardware are: logic circuits, application-specific integrated circuits (“ASICs”), large scale integrated circuits (“LSIs”), very large scale integrated circuits (“VLSIs”), and the like. Examples of configurable hardware are: one or more programmable logic devices such as programmable array logic (“PALs”), programmable logic arrays (“PLAs”), and field programmable gate arrays (“FPGAs”). Examples of programmable data processors are: microprocessors, digital signal processors (“DSPs”), embedded processors, graphics processors, math co-processors, general purpose computers, server computers, cloud computers, mainframe computers, computer workstations, and the like. For example, one or more data processors in a control circuit for a device may implement methods as described herein by executing software instructions in a program memory accessible to the processors.

Processing may be centralized or distributed. Where processing is distributed, information including software and/or data may be kept centrally or distributed. Such information may be exchanged between different functional units by way of a communications network, such as a Local Area Network (LAN), Wide Area Network (WAN), or the Internet, wired or wireless data links, electromagnetic signals, or other data communication channel.

The invention may also be provided in the form of a program product. The program product may comprise any non-transitory medium which carries a set of computer-readable instructions which, when executed by a data processor, cause the data processor to execute a method of the invention. Program products according to the invention may be in any of a wide variety of forms. The program product may comprise, for example, non-transitory media such as magnetic data storage media including floppy diskettes, hard disk drives, optical data storage media including CD ROMs, DVDs, electronic data storage media including ROMs, flash RAM, EPROMs, hardwired or preprogrammed chips (e.g., EEPROM semiconductor chips), nanotechnology memory, or the like. The computer-readable signals on the program product may optionally be compressed or encrypted.

In some embodiments, the invention may be implemented in software. For greater clarity, “software” includes any instructions executed on a processor, and may include (but is not limited to) firmware, resident software, microcode, code for configuring a configurable logic circuit, applications, apps, and the like. Both processing hardware and software may be centralized or distributed (or a combination thereof), in whole or in part, as known to those skilled in the art. For example, software and other modules may be accessible via local memory, via a network, via a browser or other application in a distributed computing context, or via other means suitable for the purposes described above.

Software and other modules may reside on servers, workstations, personal computers, tablet computers, and other devices suitable for the purposes described herein.

### Non-Liming Aspects of the Invention

The invention provides a number of non-limiting aspects. Non-limiting aspects of the invention comprise:
1. A method for detecting a presence of at least one constituent of an analyte gas based on Raman scattering, the method comprising:
   mixing a buffer gas with the analyte gas;
   exciting the mixture with a laser excitation beam;
   detecting scattered photons resulting from interaction between photons of the laser excitation beam and molecules of the analyte gas to thereby determine a spectral content of the scattered photons; and
   analyzing the spectral content to determine the presence of the at least one constituent.
2. The method of aspect 1 or any other aspect herein wherein an interaction pathlength between the photons of the excitation beam and the molecules of the analyte gas in the mixture is greater than an interaction pathlength between the photons of the excitation beam and the molecules of the analyte gas in an absence of the buffer gas.
3. The method of any one of aspects 1 to 2 or any other aspect herein wherein determining the presence of the at least one constituent is based on identifying at least one peak in the spectral content corresponding to the at least one constituent.
4. The method of aspect 3 or any other aspect herein wherein the magnitude of the at least one peak is greater than a magnitude of the at least one peak in the absence of the buffer gas.
5. The method of any one of aspects 1 to 4 or any other aspect herein comprising increasing the temperature of the mixture relative to a temperature of the ambient environment.
6. The method of aspect 5 or any other aspect herein wherein increasing the temperature of the mixture relative to the temperature of the ambient environment increases the average speed of molecules of the mixture compared to an average speed of the molecules of the mixture at the temperature of the ambient environment.
7. The method of any one of aspects 1 to 6 or any other aspect herein wherein the buffer gas has a buffer gas partial pressure and the analyte gas has an analyte gas partial pressure wherein the buffer gas partial pressure is different from (preferably greater than) the analyte gas partial pressure.
8. The method of aspect 7 or any other aspect herein wherein a ratio of the buffer gas partial pressure to the analyte gas partial pressure is in a range of about 0.01 to about 1000.
9. The method of aspect 7 or 8 or any other aspect herein comprising holding the analyte gas partial pressure constant and increasing the buffer gas partial pressure relative to the analyte gas partial pressure.
10. The method of any one of aspects 1 to 9 or any other aspect herein comprising mixing the buffer gas and the analyte gas in a gas cell.
11. The method of aspect 10 or any other aspect herein wherein exciting the mixture with the laser excitation beam comprises directing the laser excitation beam into the gas cell.
12. The method of any one of aspects 10 to 11 or any other aspect herein wherein the gas cell comprises a hollow-core fiber, the hollow-core fiber comprising a hollow core which permits permeation of fluid in the hollow core, and mixing the buffer gas with the analyte gas in the gas cell comprises introducing the buffer gas and the analyte gas into the hollow core of the hollow-core fiber.
13. The method of aspect 12 or any other aspect herein wherein a diameter of the hollow core is in a range of about 2 μm to about 50 μm.
14. The method of any one of aspects 12 to 13 or any other aspect herein wherein the hollow-core fiber has a longitudinal length in a range of about 0.5 m to about 20 m.
15. The method of any one of aspects 12 to 14 or any other aspect herein wherein the hollow-core fiber comprises a band-gap transmission hollow-core fiber.
16. The method of any one of aspects 12 to 15 or any other aspect herein wherein the hollow-core fiber comprises a hollow-core photonic crystal fiber.
17. The method of any one of aspects 12 to 14 or any other aspect herein wherein the hollow-core fiber comprises an anti-resonant hollow-core fiber.
18. The method of any one of aspects 12 to 17 or any other aspect herein comprising directing the laser excitation beam into the hollow core of the hollow-core fiber.
19. The method of aspect 18 or any other aspect herein wherein the laser excitation beam is confined by the hollow-core fiber to the hollow core.
20. The method of any one of aspects 1 to 19 or any other aspect herein comprising mixing the buffer gas and the analyte gas at an analyte source.
21. The method of aspect 20 or any other aspect herein wherein mixing the buffer gas and the analyte gas at the analyte source comprises supplying a preparation gas to the analyte source wherein the preparation gas comprises the buffer gas at a buffer gas mole fraction, wherein the analyte source may comprise breath (e.g. exhaled) of a human or other animal and the preparation gas is safe for inhalation by the human or other animal.
22. The method of aspect 21 or any other aspect herein wherein the buffer gas mole fraction in the preparation gas is in a range of about 75% to about 85%.
23. The method of any one of aspects 1 to 22 or any other aspect herein wherein the laser excitation beam comprises a pulsed single laser excitation beam.
24. The method of aspect 23 or any other aspect herein wherein the pulsed single laser excitation beam has a pulse duration in a range of about 0.01 ns to about 15 ns.
25. The method of any one of aspects 23 to 24 or any other aspect herein wherein the pulsed single laser excitation beam has a pulse repetition rate in a range of about 0.1 Hz to about 100 MHz.
26. The method of any one of aspects 23 to 25 or any other aspect herein wherein the pulsed single laser excitation beam has a spectral bandwidth less than 20 cm⁻¹.
27. The method of any one of aspects 1 to 26 or any other aspect herein wherein the buffer gas comprises an inert gas.
28. The method of any one of aspects 1 to 26 or any other aspect herein wherein the buffer gas comprises a noble gas.
29. The method of any one of aspects 1 to 26 or any other aspect herein wherein the buffer gas comprises Helium.
30. The method of any one of aspects 1 to 26 or any other aspect herein wherein the buffer gas comprises Nitrogen gas.
31. The method of any one of aspects 1 to 30 or any other aspect herein wherein the spectral content contains information about Raman intensity over a range of wavenumbers.
32. The method of any one of aspects 1 to 31 comprising any of the features, combinations of features and/or sub-combinations of features disclosed herein and/or in the accompanying drawings.
33. A method for detecting a presence of at least one constituent of an analyte gas based on Raman scattering, the method comprising:
supplying the analyte gas to a hollow core of at least one hollow-core micro-structured fiber;
exciting the analyte gas with a pulsed single optical excitation beam above a threshold pump power, the pulsed single optical excitation beam directed into the hollow core and confined by micro-structures of the at least one fiber to the hollow core to thereby interact with the analyte gas;
detecting scattered photons resulting from interaction between the photons of the excitation beam and the molecules of the analyte gas to thereby determine a spectral content of the scattered photons;
analyzing the spectral content to determine the presence of the at least one constituent.
34. The method of aspect 33 or any other aspect herein comprising supplying a buffer gas to the hollow core and wherein exciting the analyte gas with the excitation beam comprises exciting a mixture of the buffer gas and the analyte gas in the hollow core with the excitation beam.
35. The method of aspect 34 or any other aspect herein wherein an interaction pathlength between the photons of the excitation beam and the molecules of the analyte gas in the mixture is greater than an interaction pathlength between the photons of the excitation beam and the molecules of the analyte gas in an absence of the buffer gas.
36. The method of any one of aspect 33 to 35 or any other aspect herein wherein determining the presence of the at least one constituent is based on identifying at least one peak in the spectral content corresponding to the at least one constituent.
37. The method of aspect 36 or any other aspect herein wherein the magnitude of the at least one peak is greater than a magnitude of the at least one peak in the absence of the buffer gas.
38. The method of any one of aspects 33 to 37 or any other aspect herein comprising increasing the temperature of the mixture relative to a temperature of the ambient environment.
39. The method of aspect 38 or any other aspect herein wherein increasing the temperature of the mixture relative to the temperature of the ambient environment increases the average speed of molecules of the mixture compared to an average speed of the molecules of the mixture at the temperature of the ambient environment.
40. The method of any one of aspects 34 to 39 or any other aspect herein wherein the buffer gas has a buffer gas partial pressure in the mixture and the analyte gas has an analyte gas partial pressure in the mixture wherein the buffer gas
partial pressure is different from (preferably greater than) the analyte gas partial pressure.
41. The method of aspect 40 or any other aspect herein wherein a ratio of the buffer gas partial pressure to the analyte gas partial pressure is in a range of about 0.01 to about 1000.
42. The method of any one of aspects 40 to 41 or any other aspect herein comprising holding the analyte gas partial pressure constant and increasing the buffer gas partial pressure relative to the analyte gas partial pressure.
43. The method of any one of aspects 34 to 42 or any other aspect herein comprising mixing the buffer gas and the analyte gas in a gas cell and wherein supplying the analyte gas and the buffer gas to the hollow core comprises supplying the mixture to the hollow core.
44. The method of any one of aspects 33 to 43 or any other aspect herein wherein the hollow core permits permeation of fluids contained therein.
45. The method of any one of aspects 33 to 44 or any other aspect herein wherein a diameter of the hollow core is in a range of about 2 μm to about 50 μm.
46. The method of any one of aspects 33 to 45 or any other aspect herein wherein the hollow-core micro-structured fiber has a longitudinal length in a range of about 0.5 m to about 20 m.
47. The method of any one of aspects 33 to 46 or any other aspect herein wherein the hollow-core micro-structured fiber comprises a band-gap transmission hollow-core fiber.
48. The method of any one of aspects 33 to 47 or any other aspect herein wherein the hollow-core micro-structured fiber comprises a hollow-core photonic crystal fiber.
49. The method of any one of aspects 33 to 46 or any other aspect herein wherein the hollow-core micro-structured fiber comprises an anti-resonant hollow-core fiber.
50. The method of any one of aspects 1 to 49 or any other aspect herein wherein the laser excitation beam is confined by the hollow-core micro-structured fiber to the hollow core.
51. The method of any one of aspects 33 to 50 or any other aspect herein comprising mixing the buffer gas and the analyte gas at an analyte source and wherein supplying the analyte gas and the buffer gas to the hollow core comprises supplying the mixture from the analyte source to the hollow core.
52. The method of aspect 51 or any other aspect herein wherein mixing the buffer gas and the analyte gas at the analyte source comprises supplying a
preparation gas to the analyte source wherein the preparation gas comprises the buffer gas at a buffer gas mole fraction, wherein the analyte source may comprise breath (e.g. exhaled) of a human or other animal and the preparation gas is safe for inhalation by the human or other animal.
53. The method of aspect 52 or any other aspect herein wherein the buffer gas mole fraction in the preparation gas is in a range of about 75% to about 85%.
54. The method of any one of aspects 33 to 53 or any other aspect herein wherein the pulsed single optical excitation beam comprises a pulsed single laser excitation beam.
55. The method of aspect 54 or any other aspect herein wherein the pulsed single laser excitation beam has a pulse duration in a range of about 0.01 ns to about 15 ns.
56. The method of any one of aspects 54 to 55 or any other aspect herein wherein the pulsed single laser excitation beam has a pulse repetition rate in a range of about 0.1 Hz to about 100 MHz.
57. The method of any one of aspects 54 to 56 or any other aspect herein wherein the pulsed single laser excitation beam has a spectral bandwidth less than 20 cm⁻¹.
58. The method of any one of aspects 34 to 57 or any other aspect herein wherein the buffer gas comprises an inert gas.
59. The method of any one of aspects 34 to 57 or any other aspect herein wherein the buffer gas comprises a noble gas.
60. The method of any one of aspects 34 to 57 or any other aspect herein wherein the buffer gas comprises Helium.
61. The method of any one of aspects 34 to 57 or any other aspect herein wherein the buffer gas comprises Nitrogen gas.
62. The method of any one of aspects 33 to 61 or any other aspect herein wherein the spectral content contains information about Raman intensity over a range of wavenumbers.
63. The method of any one of aspects 33 to 62 comprising any of the features, combinations of features and/or sub-combinations of features disclosed herein and/or in the accompanying drawings.
64. A system for detecting a presence of at least one constituent of an analyte gas based on Raman scattering, the system comprising:
   a gas cell for holding gas;
   one or more gas supplies in fluid connection with the gas cell for supplying the analyte gas and a buffer gas to the gas cell;
   an optical excitation source optically connected to the gas cell for emitting an optical excitation beam into the gas cell, the optical excitation beam propagating through the gas cell to interact with the analyte gas;
   a detector optically connected to the gas cell for detecting scattered photons resulting from interaction between the excitation beam and the analyte gas to thereby determine a spectral content of the scattered photons; and,
   a processor in communication with the detector for analyzing the spectral content to determine the presence of the at least one constituent.
65. The system of aspect 64 comprising any of the features, combinations of features and/or sub-combinations of features of any of the other aspects recited herein.
66. The system of any one of aspects 64 to 65 comprising any of the features, combinations of features and/or sub-combinations of features disclosed herein and/or in the accompanying drawings.
67. A system for detecting a presence of at least one constituent of an analyte gas based on Raman scattering, the system comprising:
   at least one hollow-core micro-structured fiber, the at least one fiber comprising a hollow core;
   an analyte gas supply in fluid connection with the at least one fiber for supplying the analyte gas to the hollow core;
   an optical excitation source optically connected to the at least one fiber for emitting a pulsed single optical excitation beam into the hollow core above a threshold pump power, the pulsed single optical excitation beam confined by micro-structures of the at least one fiber to the hollow core to thereby interact with the analyte gas therein;
   a detector optically connected to the at least one fiber for detecting scattered photons resulting from interaction between photons of the pulsed single excitation beam and molecules of the analyte gas; and,
   a processor in communication with the detector for analyzing the spectral content to determine the presence of the at least one constituent.
68. The system of aspect 67 comprising any of the features, combinations of features and/or sub-combinations of features of any of the other aspects recited herein.
69. The system of any one of aspects 67 to 68 comprising any of the features, combinations of features and/or sub-combinations of features disclosed herein and/or in the accompanying drawings.
70. Apparatus having any new and inventive feature, combination of features, or sub-combination of features as described herein.
71. Methods having any new and inventive steps, acts, combination of steps and/or acts or sub-combination of steps and/or acts as described herein.

### INTERPRETATION OF TERMS

Unless the Context Clearly Requires Otherwise, Throughout the Description and the claims:
“comprise”, “comprising”, and the like are to be construed in an inclusive sense, as opposed to an exclusive or exhaustive sense; that is to say, in the sense of “including, but not limited to”;
“connected”, “coupled”, or any variant thereof, means any connection or coupling, either direct or indirect, between two or more elements; the coupling or connection between the elements can be physical, logical, or a combination thereof;
“herein”, “above”, “below”, and words of similar import, when used to describe this specification, shall refer to this specification as a whole, and not to any particular portions of this specification;
“or”, in reference to a list of two or more items, covers all of the following interpretations of the word: any of the items in the list, all of the items in the list, and any combination of the items in the list;
the singular forms “a”, “an”, and “the” also include the meaning of any appropriate plural forms. These terms (“a”, “an”, and “the”) mean one or more unless stated otherwise;
“and/or” is used to indicate one or both stated cases may occur, for example A and/or B includes both (A and B) and (A and B);
“approximately” when applied to a numerical value means the numerical value±10%;
where a feature is described as being “optional” or “optionally” present or described as being present “in some embodiments” it is intended that the present disclosure encompasses embodiments where that feature is present and other embodiments where that feature is not necessarily present and other embodiments where that feature is excluded. Further, where any combination of features is described in this application this statement is intended to serve as antecedent basis for the use of exclusive terminology such as “solely,” “only” and the like in relation to the combination of features as well as the use of “negative” limitation(s)” to exclude the presence of other features; and
“first” and “second” are used for descriptive purposes and cannot be understood as indicating or implying relative importance or indicating the number of indicated technical features. Words that indicate directions such as “vertical”, “transverse”, “horizontal”, “upward”, “downward”, “forward”, “backward”, “inward”, “outward”, “left”, “right”, “front”, “back”, “top”, “bottom”, “below”, “above”, “under”, and the like, used in this description and any accompanying claims (where present), depend on the specific orientation of the apparatus described and illustrated. The subject matter described herein may assume various alternative orientations. Accordingly, these directional terms are not strictly defined and should not be interpreted narrowly.

Where a range for a value is stated, the stated range includes all sub-ranges of the range. It is intended that the statement of a range supports the value being at an endpoint of the range as well as at any intervening value to the tenth of the unit of the lower limit of the range, as well as any subrange or sets of sub ranges of the range unless the context clearly dictates otherwise or any portion(s) of the stated range is specifically excluded. Where the stated range includes one or both endpoints of the range, ranges excluding either or both of those included endpoints are also included in the invention.

Certain numerical values described herein are preceded by “about”. In this context, “about” provides literal support for the exact numerical value that it precedes, the exact numerical value ±5%, as well as all other numerical values that are near to or approximately equal to that numerical value. Unless otherwise indicated a particular numerical value is included in “about” a specifically recited numerical value where the particular numerical value provides the substantial equivalent of the specifically recited numerical value in the context in which the specifically recited numerical value is presented. For example, a statement that something has the numerical value of “about 10 ” is to be interpreted as: the set of statements:
in some embodiments the numerical value is 10;
in Some Embodiments the Numerical Value Is in the Range of 9.5 to 10.5;
   and if from the context the person of ordinary skill in the art would understand that values within a certain range are substantially equivalent to 10 because the values with the range would be understood to provide substantially the same result as the value 10 then “about 10” also includes:
in some embodiments the numerical value is in the range of C to D where C and D are respectively lower and upper endpoints of the range that encompasses all of those values that provide a substantial equivalent to the value 10

Specific examples of systems, methods and apparatus have been described herein for purposes of illustration. These are only examples. The technology provided herein can be applied to systems other than the example systems described above. Many alterations, modifications, additions, omissions, and permutations are possible within the practice of this invention. This invention includes variations on described embodiments that would be apparent to the skilled addressee, including variations obtained by: replacing features, elements and/or acts with equivalent features, elements and/or acts; mixing and matching of features, elements and/or acts from different embodiments; combining features, elements and/or acts from embodiments as described herein with features, elements and/or acts of other technology; and/or omitting combining features, elements and/or acts from described embodiments.

As will be apparent to those of skill in the art upon reading this disclosure, each of the individual embodiments described and illustrated herein has discrete components and features which may be readily separated from or combined with the features of any other described embodiment(s) without departing from the scope of the present invention.

Any aspects described above in reference to apparatus may also apply to methods and vice versa.

Any recited method can be carried out in the order of events recited or in any other order which is logically possible. For example, while processes or blocks are presented in a given order, alternative examples may perform routines having steps, or employ systems having blocks, in a different order, and some processes or blocks may be deleted, moved, added, subdivided, combined, and/or modified to provide alternative or subcombinations. Each of these processes or blocks may be implemented in a variety of different ways. Also, while processes or blocks are at times shown as being performed in series, these processes or blocks may instead be performed in parallel, simultaneously or at different times.

Various features are described herein as being present in “some embodiments”. Such features are not mandatory and may not be present in all embodiments. Embodiments of the invention may include zero, any one or any combination of two or more of such features. All possible combinations of such features are contemplated by this disclosure even where such features are shown in different drawings and/or described in different sections or paragraphs. This is limited only to the extent that certain ones of such features are incompatible with other ones of such features in the sense that it would be impossible for a person of ordinary skill in the art to construct a practical embodiment that combines such incompatible features. Consequently, the description that “some embodiments” possess feature A and “some embodiments” possess feature B should be interpreted as an express indication that the inventors also contemplate embodiments which combine features A and B (unless the description states otherwise or features A and B are fundamentally incompatible). This is the case even if features A and B are illustrated in different drawings and/or mentioned in different paragraphs, sections or sentences.

It is therefore intended that the following appended claims and claims hereafter introduced are interpreted to include all such modifications, permutations, additions, omissions, and sub-combinations as may reasonably be inferred. The scope of the claims should not be limited by the preferred embodiments set forth in the examples, but should be given the broadest interpretation consistent with the description as a whole.

What is claimed is:

1. A method for detecting a presence of at least one constituent of an analyte gas based on Raman scattering, the method comprising:
mixing a buffer gas with the analyte gas;
exciting the mixture with a laser excitation beam;
detecting scattered photons resulting from interaction between photons of the laser excitation beam and molecules of the analyte gas to thereby determine a spectral content of the scattered photons; and
analyzing the spectral content to determine the presence of the at least one constituent.

2. The method of claim 1 wherein an interaction pathlength between the photons of the excitation beam and the molecules of the analyte gas in the mixture is greater than an interaction pathlength between the photons of the excitation beam and the molecules of the analyte gas in an absence of the buffer gas.

3. The method of claim 1 wherein determining the presence of the at least one constituent is based on identifying at least one peak in the spectral content corresponding to the at least one constituent.

4. The method of claim 3 wherein the magnitude of the at least one peak is greater than a magnitude of the at least one peak in the absence of the buffer gas.

5. The method of claim 1 comprising increasing the temperature of the mixture relative to a temperature of the ambient environment.

6. The method of claim 5 wherein increasing the temperature of the mixture relative to the temperature of the ambient environment increases the average speed of molecules of the mixture compared to an average speed of the molecules of the mixture at the temperature of the ambient environment.

7. The method of claim 1 wherein the buffer gas has a buffer gas partial pressure and the analyte gas has an analyte gas partial pressure wherein the buffer gas partial pressure is different from (preferably greater than) the analyte gas partial pressure.

8. The method of claim 7 wherein a ratio of the buffer gas partial pressure to the analyte gas partial pressure is in a range of about 0.01 to about 1000.

9. The method of claim 7 comprising holding the analyte gas partial pressure constant and increasing the buffer gas partial pressure relative to the analyte gas partial pressure.

10. The method of claim 1 comprising mixing the buffer gas and the analyte gas in a gas cell.

11. The method of claim 10 wherein exciting the mixture with the laser excitation beam comprises directing the laser excitation beam into the gas cell.

12. The method of claim 10 wherein the gas cell comprises a hollow-core fiber, the hollow-core fiber comprising a hollow core which permits permeation of fluid in the hollow core, and mixing the buffer gas with the analyte gas in the gas cell comprises introducing the buffer gas and the analyte gas into the hollow core of the hollow-core fiber.

13. The method of claim 12 wherein a diameter of the hollow core is in a range of about 2 μm to about 50 μm.

14. The method of claim 12 wherein the hollow-core fiber has a longitudinal length in a range of about 0.5 m to about 20 m.

15. The method of claim 12 wherein the hollow-core fiber comprises a band-gap transmission hollow-core fiber.

16. The method of claim 12 wherein the hollow-core fiber comprises a hollow-core photonic crystal fiber.

17. The method of claim 12 wherein the hollow-core fiber comprises an anti-resonant hollow-core fiber.

18. The method of claim 12 comprising directing the laser excitation beam into the hollow core of the hollow-core fiber.

19. The method of claim 18 wherein the laser excitation beam is confined by the hollow-core fiber to the hollow core.

20. The method of claim 1 comprising mixing the buffer gas and the analyte gas at an analyte source.
